(19) United States
(12) Patent Application Publication
(10) Pub. No.: US 2004/0138602 A1
(43) Pub. Date: Jul. 15, 2004

(54) Simplified one-handed preemptive medical procedure site dressing to prevent sharps injuries and exposure to bloodborne pathogens

(76) Inventor: Joel S. Rossen, Tamarac, FL (US)

Correspondence Address: JOHN F. SALAZAR, MIDDLETON & REUTLINGER, 2500 BROWN & WILLIAMSON TOWER, LOUISVILLE, KY, 40202, US

(21) Appl. No.: 10/696,041

(22) Filed: Oct. 29, 2003

Related U.S. Application Data
(60) Provisional application No. 60/422,292, filed on Oct. 30, 2002.
(60) Provisional application No. 60/499,118, filed on Aug. 29, 2003.

(51) Int. Cl. A61F 13/00

(52) U.S. Cl. 602/41

(57) ABSTRACT

A dressing for use with medical procedures is provided that permits application of the dressing prior to the commencement of a medical procedure and wherein the dressing remains on the site after the procedure has been completed. The dressing comprises an absorbent pad, a hinged door and an aperture through which the procedure site can be accessed or it comprises an adhesive bandage part, which is bent and secured in such a way that the bandage forms a door and a hinge, and an adhesive flap component which anchors the bandage to the skin. The bandage is positioned in such a way as to infer or define the location where the procedure is to be performed. In either embodiment, when it is deployed, the door or bandage is rotated around the hinge and can be set in place at a procedure location prior to the procedure. Importantly, the device can be deployed with one hand, to complete the bandaging of a procedure site simultaneous with or within a moment after the completion of the medical procedure. The device is not size-limited by the disclosure. The invention may be small enough to use to perform an injection or it may be large enough that a major surgical procedure can be performed through the aperture. Methods of using the dressing of the present invention are also provided.

### CROSS-REFERENCE TO RELATED APPLICATIONS

This nonprovisional application is a continuation of provisional application serial No. 60/422,292, filed Oct. 30, 2002, and a continuation of provisional application serial No. 60/499,118, filed Aug. 29, 2003.

### BACKGROUND OF THE INVENTION

1. Field of the Invention

The present invention relates to dressings for broken skin, such as ulcers, surgical interventions, vaccinations, or needle puncture sites on humans or animals, which permit application of the dressing in compliance with needlestick prevention guidelines and further provides access to the lesion without removal of the dressing. More specifically, this invention relates to the facilitation of one-handed application of such dressings by providing a dressing and method whereby the dressings can be placed on the procedure site prior to the performance of an invasive medical procedure.

2. Description of the Related Art

Healthcare workers are at risk of serious infections if exposed to pathogens which are commonly present on needles and other sharp devices after such devices are used to break, cut, or puncture the skin of a patient. It is widely recognized that body fluids containing bloodborne pathogens are a serious vector of transmission of infectious diseases. The spread of bloodborne pathogens to clinical practitioners and others by contact with the body fluids of an infected patient is an inherent risk that is routinely taken when conducting procedures involving skin punctures and releasing blood and other body fluids. Further, certain procedures, such as live-virus vaccinations, require inoculation with materials that may themselves be potentially infectious.

As such, numerous protocols and medical devices have been developed in order to minimize risks. In particular, safe needle handling, needle disposal practices, needle covers, and needle retractors help to prevent inadvertent needle sticks with contaminated sharps. Puncture site coverings and wound coverings protect others from contact with post-procedure exudate. Numerous types of absorbent and adhesive bandages are known in the art that can be applied to a puncture site or vaccination site on a patient. In general, these bandages include an absorbent material that covers the procedure site and an adhesive to keep the absorbent material in place and, in some cases, to isolate it. The bandages both protect the patient from microbial contamination of the broken skin while healing and also protect practitioners from the body fluids that can shoot, spray, or seep from the wounds. Although traditional bandages perform these functions, to a certain extent, they do not offer the advantages that accompany rapid deployment nor do they offer needle stick injury protection and shielding of microbial contamination during such a procedure. Further, in the prior art, it has not been possible to complete covering a procedure site prior to, or within moments of, completing an invasive procedure, because one hand was generally occupied performing the procedure while the other was occupied disposing of the contaminated sharp.

Therefore, it would be advantageous to have a procedure site dressing available to clinicians that overcame the above-cited disadvantages. In particular, it would be desirable to have available a dressing that permitted the clinician to apply the dressing before performing an invasive procedure so that contaminated sharps could be discarded without the clinician having to simultaneously bandage the site. It would also be advantageous to have a dressing that permitted the clinician to intermittingly monitor and access the procedure site over time without requiring removal of the covering.

### SUMMARY OF THE INVENTION

In view of the known deficiencies associated with earlier dressings, the present invention facilitates one-handed application of the dressing by providing a dressing and method whereby the dressing can be placed on the procedure site prior to the performance of an invasive medical procedure. When used prior to an injection or a vaccination procedure, such a device would decrease the time the person performing the medical service would be in possession of a contaminated sharp by allowing the completion of the bandaging procedure, using one hand, within a fraction of a second of completing the procedure. A device of this nature is in alignment with the intent of current needle stick injury prevention guidelines.

Because traditional bandages were developed when third party exposure to needle stick injuries and bloodborne pathogens was less of a concern, methods for using unique bandaging techniques to aid in the prevention and transmission of microbial exposure during an invasive procedure were not and did not then need to be incorporated into their design. By today's standards, traditional bandages are no longer adequate to protect healthcare workers from exposure to bloodborne pathogens because they are first applied after vaccinations, inoculations, injections, surgeries, or other such invasive medical procedures. Because traditional bandages are applied over the procedure site after the procedure, they therefore require manipulation of the site and the bandage, including wiping blood and other fluids from the procedure site, removing any backing from the bandage, and properly securing the bandage to the site after such a procedure is completed. These necessary functions must all be performed while the medical practitioner is maintaining possession of or is attempting to simultaneously dispose of a contaminated needle or other sharp instrument. Otherwise, the medical practitioner must leave the site unattended while disposing of the contaminated sharp and then return to bandage the puncture site. This creates a hazardous scenario in that it becomes more likely that practitioners may either stick themselves, or others, with the sharp or contaminate themselves with the patient's body fluids while their attention is divided among these conflicting tasks or that the patient may touch and contaminate the procedure site while the practitioner is focused on proper sharps disposal.

The present invention is for an invasive medical procedure site dressing for use on humans or animals that includes an absorbent layer contacting the skin and defining an aperture through which the medical procedure can be performed. The absorbent layer absorbs fluids at the procedure site created by exudate from the wound and excess material from the procedure. Hingedly affixed to an upper surface of the absorbent layer and covering the aperture is a flexible door that protects the site after the procedure. The door may be transparent so that the site can be visually monitored as it heals or an expected reaction develops.

By providing a bandage that is placed over or near the procedure site prior to commencing the invasive part of the procedure, the bandage itself becomes part of the procedure's process and can be used to complete the bandaging process within moments of or synchronous with completing the procedure. This new syntax frees the provider to dispose of the contaminated sharp immediately, rather than after having maintained possession of the contaminated sharp while simultaneously performing a bandaging procedure on the site with the other hand. The site dressing, along with the method for bandaging a medical procedure site prior to performing the procedure, decreases the healthcare provider', s exposure to bloodborne pathogens. Furthermore, when the invention is being used in this surgical drape configuration, duplication of materials is prevented by permitting the surgical drape to double as the post-surgical bandage.

The dressing of the present invention includes a pull-tab, a hinge, an adhesive coated material, and optionally, an absorbent pad. The invention is a bandage that has at least one part that adheres to skin prior to a medical procedure. Once placed, the bandage remains clear of the operative field until it is needed. The section of the bandage that contacts the skin either has a portion removed to define an aperture where a procedure is to be performed or it is designed and configured in such a way that it can be placed close to the procedure site so as to infer the procedure's intended location from the positioning of specific components of the bandage. The present invention provides for a dressing that can be applied over, or near to, an invasive procedure site, prior to commencing the procedure. The present invention also provides for a dressing that can remain on, at, or near to the site during the procedure and can cover the site almost instantly after the procedure has been completed. Further, the present invention provides for a dressing that protects an invasive procedure site from contamination while it heals. Also, the present invention provides for a bandage that can cover a procedure site within moments of the completion of the procedure using only one hand, that can be used preemptively to prevent exposure of healthcare workers to bloodborne pathogens, and that can be re-opened to observe, evaluate, service, clean, or treat the site. The bandage will shield the healthcare provider from aerosols, sprays, and leakages of potentially contaminated fluids during the course of the procedure. Also, the present invention provides for a bandage that can not only be applied prior the performance of an invasive procedure, but that can provide access later to that invasive site for observation or treatment.

The invasive medical procedure site dressing for use on humans or animals may include an absorbent layer or pad which may or may not contact the skin and which either defines the procedure site via an aperture in the bandage through which the medical procedure can be performed or is adhered to the skin in such a position on the body that the location of the procedure site is easily inferred from the shape or position of the bandage on the skin. If present, the absorbent layer absorbs the blood and/or the body fluids at the procedure site created by exudate, bleeding, or seepage from the wound or puncture hole at the procedure site.

The invention is comprised of a flexible, adhesive-coated material, which forms a door or a base layer flap and is hingedly affixed to the skin. Such base layer may be a simple strip of adhesive coated material, or it may be large enough to cover and surround the procedure site, even a major surgical site. The invention may be formed with the addition of a through and through aperture so it can be positioned in such a way as to fully surround the procedure site prior to the inception of a procedure such as an injection or a surgery procedure so it can remain in place to protect the site after the procedure is completed. It may have a layer of absorbent material between the base and the skin and surrounding the procedure site with the adhesive layer of the base bandaging material extended beyond the edges of the absorbent layer to make contact with the skin 360-degrees around the absorbent layer and hence encircle and protect the lesion. Such absorbent layer would, in many embodiments, have a through and through aperture coincident with the aperture of the base bandaging material. The absorbent layer could, in fact, lack an aperture or its aperture could be smaller or larger than that of the base-bandaging layer. The door may be transparent so that the site can be visually monitored as it heals or as an expected reaction develops. The observation door made at least in part of a transparent flexible material may be hingedly affixed to a top surface of the patch base layer and may be provided in its open conformation as packaged.

If present, the aperture may be comprised of a material that limits the movement of bodily fluids, such as a closed cell foam, so as to prevent the fluids from escaping from the operative field or procedure site. The bandage may be of a size small enough to protect a patient and operator during a venous or arterial blood draw, vaccination, or injection procedure, or it may be large enough to perform the function of a surgical drape. Additionally, the through and through aperture may be covered or otherwise completely obscured by a material film, such as a thermoplastic elastomer, which is typically clear or translucent, non-coring, non-pyrogenic, and self-sealing. Such a film could be punctured by a needle or medical device during a procedure such as an injection and it would re-seal itself to keep body fluids within the cavity formed by the bandage and the film and the skin. By virtue of its self-sealing properties, such a membrane would provide an additional level of protection to the clinician from exposure to bloodborne pathogens and body fluids.

In one embodiment, the base layer may be absent completely and the hinge can be formed by a bend in the flexible door itself, which is now little more than a simple bandage which is fitted with the deployment method of the invention. In this embodiment, one side of the adhesive surface of the door would be affixed directly to the skin and a hinge would be formed out of much of the rest of the bandage. A piece of tape or flexible material, such as, but not limited to, polyethylene, polyurethane, or vinyl, which may be coated with adhesive on one or both sides, can be used as the hinge. While the material is flexible and can be folded to use as a hinge, it is sufficiently rigid and springy so that it will maintain its shape, even under the burden of the full weight of the door (i.e., the body of the bandage), and it will hold the door open, when otherwise unstressed. This hinge material adhesively affixes the door to the bandage-body and holds the door open by functioning as a spring to keep the door open until such time as the door is deployed (i.e., closed) and the door's adhesive contacts the base of the bandage. The weight or flexibility of the door of this embodiment may be such that an additional adhesive may be needed on the back of the body of the bandage to temporarily adhere the top of the bandage's body of the bandage to the skin until the device is deployed. When closed, the adhesion of the door to the base or to the skin is greater than the strength or recoil of the hinge-spring and hence, the door stays closed. Also, the door may be held in its open position with a releasable adhesive bead located on the patch base layer.

In another embodiment, instead of a separate hinge, one edge of the door might be folded under in such a way that the adhesive of the door contacts the base and the release paper holds the door in place by an overlapping fold or second hinge which blocks the hinge from rotating but still allows the front section of the release paper to rotate and bring the absorbent pad down to the aperture. In yet another similar embodiment, the adhesive that holds the door and the base together when the device is closed (i.e., deployed) is on the base layer, rather than the door. In still yet another embodiment, the base layer is absent and the hinge material is adhered directly to the skin and holds the door open. If the door (or bandage or body, as the case may be) is too heavy, a bit of adhesive between the back of the bandage and the skin, to hold the bandage in its un-deployed configuration, may be necessary.

An important function of the door is that it remains clear of the operative field during the procedure. This can be accomplished in a number of ways and hence altering the way in which the door is kept clear of the operative field can produce numerous embodiments of the invention without diverging from the scope of the invention. In one embodiment, the door is attached to the base by a separate flexible piece of adhesive coated tape that can fold and function as a hinge. This tape has enough flexibility for the door to be closed completely and enough rigidity to keep it open during the procedure and until needed. For example, the tape may be an adhesive coated polyethylene sheet. Closing the door brings its adhesive coating into contact with the skin or with the exposed surface of the base layer. When the adhesive makes contact with the skin or base surface, the adhesive overcomes the spring-tension of the hinge and holds it closed.

When closed, the observation door will cover the aperture. The observation door may further have attached in its top surface (when open) an absorbent pad sized to fill the aperture when the observation door is closed. This permits absorption of excess fluids at the site post-procedure. The absorbent pad may be removed from the door and discarded after use. The observation door is then closed again to protect the site from contamination. In a further alternative embodiment, the observation door may be opaque so that the site is not readily viewable by the casual observer. Alternatively, a second opaque outer door may be affixed over the observation door. Thus, the outer door blocks casual viewing of the procedure site, but can be easily lifted by the clinician for viewing of the site through the observation door.

In another embodiment, virtually the entire device is equivalent to the door and it is held in place by adhesives that limit the movement of the body of the bandage. In this embodiment, there is no separate base layer. This embodiment is essentially a simple bandage that is folded such that it incorporates the methods of this application to remain folded and ready to deploy near a procedure site so it can be used preemptively as described herein.

The optional absorbent pad provides protection from exposure to bloodborne pathogens for a person giving an injection. To this end, the device is designed so it can be easily and rapidly deployed during an injection procedure, even before the needle has been fully removed from the patient. The embodiments designed for use with needle punctures include a flexible absorbent pad which is positioned in such a way that after the puncture has taken place and while the door is being closed, the flexible absorbent pad can easily be bent and brought into contact with the needle-skin interface. In this way, the invention performs the traditional function of covering the needle as it is removed to prevent spray or aerosol of body fluids and it puts pressure on the injection or blood draw site to help close the wound.

Since all skin puncture procedures should be performed using universal precautions, in a typical embodiment of this invention, the body of this device is made from materials which prevent the passage of blood, body fluids, and pathogens through from the lesion site to the outside surfaces of the bandage where someone could come in contact with the pathogens. For example, a thin layer of polyurethane is impervious to bacteria and viruses. Optionally, the body of the device potentially could be constructed from porous materials, if the specific function of preventing organic materials from reaching the surface were not an issue. A bandage of this type might be used for simple cuts and scrapes on healthy children, for example.

In another embodiment, perhaps as packaged for sale, the absorbent layer is sandwiched between a bottom coated release-paper carrier that is removed immediately prior to using the dressing, and a base layer of flexible material with an adhesive coated bottom surface for attaching the dressing to the procedure site. The absorbent layer and patch base layer each define a procedure site aperture so that access to the procedure site is possible through both layers. In this embodiment, the adhesive of the patch base layer would typically extend beyond and surround the periphery of the absorbent layer so the adhesive of the base layer would envelop the absorbent layer and seal all absorbent materials and fluids and pathogens within the confines of the bandage.

In yet another embodiment, the invention is a simple bandage comprised of a strip of tape with a piece of absorbent material adhered, and generally centered, to its adhesive side, similar to the plastic bandaging strips commonly used for small cuts. This device would be folded and fitted with a mechanism that could hold it together such that one could adhere it to the skin in the folded configuration, ready to deploy, on the skin near a procedure site, such as an injection site. To deploy the device means, ‘to close the bandage over and against the procedure site.’ The retention mechanism could be a straight or folded piece of release-paper, typically coated for adhesive release on one side only, with the coated side facing the adhesive of the bandage and which may have a small piece of adhesive or adhesive coated material or adhesive coated film adhered to the non-release side of the release-paper or release paper with a section removed.

In yet another embodiment, the release-paper has no adhesive coating. Here, the release-paper has a section or sections removed which allows the adhesive from one portion of the bandage, which is otherwise covered by the release coated surface of the release-paper mechanism, to extend through or past the surface of the release-paper and adhere to the non-adhesively coated back of the other end of that same bandaging material and hence, hold the device in its locked and ready configuration until deployed. The materials of the bandage and release paper are very thin, and relatively small discontinuances in the release-paper easily permit the adhesive of the bandage to pass through the release-paper to contact the non-adhesive back of the bandage and hold the embodiment cocked. One end of the release-paper can form a pull-tab to deploy the device and can be positioned in such a way that a section of the adhesive side of the body of the bandage can hold the bandage in place against the skin. The release-paper, in turn, is temporarily releasably adhered to the adhesive of the bandage strip. The adhesive coated or fenestrated end of the release-paper strip is positioned within the fold of the bandage against the back of the bandage in two places. Hence, using any of the preceding methods, the hinge is maintained in its fully open position until the release-paper tab is pulled and the release-paper is pulled free from its attachment(s) to the bandage. With the adhesive coated version of the release paper, once the adhesive coated end of the release-paper is pulled free of one side of the folded back of the bandage, the bandage is freed to rotate on its hinge in such a way that the bandage can be closed over the procedure site very quickly.

In yet another embodiment, there is an absorbent material positioned on the bandage in such a way that the absorbent material will cover the procedure site when the invention is deployed. The absorbent material may be fully circumscribed by the adhesive layer in such a way that when the device is deployed, a ring of adhesive coated material completely surrounds the absorbent material so as to surround the procedure site, and seal it within a ring of bandaging material. Else, the absorbent material may traverse the full width of the body of the bandage and hence, the procedure site would be covered by absorbent padding material. In this case, the procedure site would be covered, but it would not be completely isolated by an adhesive seal after the device was deployed.

In yet another embodiment, the site dressing has an aperture in the base and an observation door, which may be made in part of a transparent, stiff, or flexible material, and is hingedly affixed to a top surface of the patch base layer and is provided in its “open” configuration as packaged. When closed, the transparent door, or a transparent part of the door, will cover the aperture and the procedure site, and will allow observation of the site without requiring removal of the bandage. The door may further have attached on its top surface (when open) an absorbent pad, cut to size to either fill the aperture or cover, without entering the aperture, when the door is closed. The door, whether transparent to permit ongoing site observation or opaque so as to obscure the site, may be closable and re-openable so as to permit repeated access to the site for observation or treatment. Such a function can be achieved by using a non-permanent re-positionable adhesive on the adhesive side of the door and may be enhanced by adding a non-adhesively coated tab to the edge of the door to assist in opening the door.

In the embodiment in which the pad fills the aperture, the pad assists post-procedure absorption of excess fluids at the site. Additionally, in the embodiment in which the pad covers but does not fill nor enter the aperture or one in which the pad enters the aperture but is not as thick as the base, and hence does not contact the skin when the door is closed, the pad and aperture form a chamber in which the procedure site can be enclosed, although the site itself is not actually touched by any of the bandaging materials. The absorbent pad may be attached to the adhesive surface of the door of the bandage, in which case it will remain with the device when it has been closed or it may be attached to the release-paper so that it can be disposed of immediately after the procedure has been completed when the release-paper, pull-tab closer/deployer component is removed from the device to fully deploy it. The absorbent pad can also be backed by a release paper layer, essentially the size of the pad, so it can be removed from the door and discarded. These embodiments would be useful for burns or for vaccinations, such as smallpox vaccinations, where blotting the site could be useful.

In another embodiment, a piece of release material can be adhered to the adhesive surface of the door, such that pulling the tab would rotate the door on its hinge to close the door over the procedure site aperture with one single movement. In other alternative embodiments, the door can be clear, translucent or opaque, depending on whether, in the intended use, it is desirable for the procedure site to be readily viewable and it can be re-openable, or not.

The present invention further includes methods of applying dressings to human or animal tissue prior to the inception of an invasive procedure, such as an injection. The present invention includes methods of retaining the bandage in an un-deployed state and preventing it from prematurely entering the operative field. The invention provides a method for configuring bandages that can be positioned over or near a proposed invasive medical procedure site prior to the inception of the procedure. A base layer of the bandage is adhered adhesively to the skin and the design of the bandage keeps all parts of the bandage out of the operative field so that the procedure can be performed unimpeded. During the final moments of the procedure or as soon as the procedure is completed, the bandage can be rapidly deployed with one hand. The present invention provides for a method of applying bandages to a site, prior to the inception of an invasive procedure, and a method of preventing potentially obstructive parts of the invention from entering or obscuring the view of the operative field until the procedure is completed and the device is to be deployed.

Certain methods of folding, taping, placing, and deploying the bandages also disclosed in this application will greatly reduce the time it takes to complete the bandaging of a procedure site after the procedure has been performed. One purpose of the invention is to shorten the time a healthcare provider is in possession of a contaminated sharp by decreasing the time it takes to complete the one-handed bandaging of an invasive procedure site once the procedure has been completed. Furthermore, because the bandage of the present invention is applied before the procedure is performed, not after performing a puncture, healthcare providers need not choose between disposing of a contaminated sharp, thus protecting themselves, and bandaging the site while maintaining possession of the contaminated sharp, thus protecting their patient. The surgical patient is protected from the potential infections that could occur when a fresh surgical site is unnecessarily handled during an immediate post-surgical bandaging procedure that could have otherwise been avoided by simply closing the window on the drape immediately after the surgical procedure is completed.

For a better understanding of the present invention, together with other and further objects thereof, reference is made to the following description, taken in conjunction with the accompanying drawings, wherein like numerals refer to like elements throughout the several views, and its scope will be pointed out in the appended claims.

### BRIEF DESCRIPTION OF THE DRAWINGS

FIG. 1 is a top view of an embodiment of the present invention in its open configuration.

FIG. 2 is a top view showing the disassembled components making up the embodiment of FIG. 1.

FIG. 3 is a side view showing the disassembled components making up the embodiment of FIG. 1.

FIG. 4a is a side view of the embodiment shown in FIG. 1 in its open configuration.

FIG. 4b is a side view of the embodiment shown in FIG. 1 after use in its closed configuration.

FIG. 5a is a side view of an embodiment of the present invention in its open configuration.

FIG. 5b is a side view of the embodiment of FIG. 5a of the present invention after use in its closed configuration.

FIG. 6 is a perspective view of an embodiment of the present invention.

FIG. 7 is a top view of an embodiment of the dressing of the present invention in an open configuration.

FIG. 8 is a top view of the disassembled components of the embodiment of FIG. 7 of the present invention.

FIG. 9 is a side view of the disassembled components of the embodiment of FIG. 7 of the present invention.

FIG. 10 is a side view of the embodiment of FIG. 7 of the present invention in a closed configuration.

FIG. 10a is an oblique top view the embodiment of FIG. 7 of the present invention in a partially closed configuration.

FIG. 10b is an oblique bottom view of the embodiment of FIG. 7 of the present invention in a partially closed configuration.

FIG. 10c is an oblique side view of an embodiment of the dressing of the present invention in a partially closed configuration.

FIG. 10d is an oblique top view of the embodiment of FIG. 10c of the present invention in a partially closed configuration.

FIG. 10e is an oblique top view of an embodiment of the dressing of the present invention in a partially closed configuration.

FIG. 10f is a partially disassembled view of the embodiment of FIG. 10e of the present invention.

FIG. 10g is an oblique top view of an embodiment of the dressing of the present invention.

FIG. 11 is a top view of an embodiment of the deployer and dressing of the present invention.

FIG. 11a is a bottom view of the embodiment of FIG. 111 of the present invention.

FIG. 11b is a bottom view of an embodiment of the deployer and dressing of the present invention.

FIG. 11c shows the first step in a folding sequence of the embodiment of FIG. 11b of the present invention.

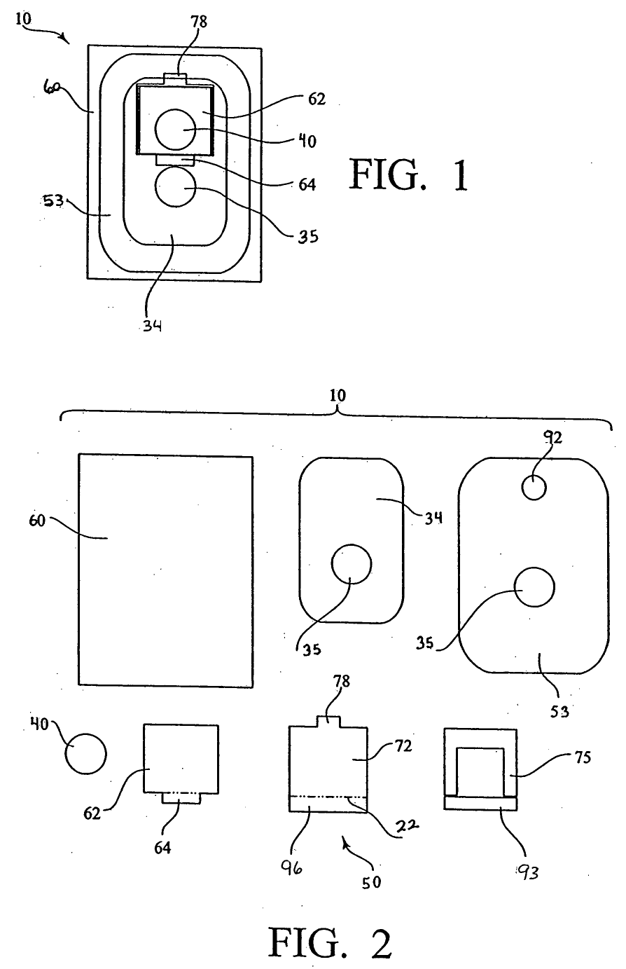

FIG. 1d shows the second step in the folding sequence of the embodiment of FIG. 111b of the present invention.

FIG. 12 is a side view of an embodiment of the deployer and dressing of the present invention.

FIG. 12a is a folded view of the embodiment of FIG. 12 of the present invention.

FIG. 12b is an unfolded oblique view of the embodiment of FIG. 12 of the present invention.

FIG. 13 is a side view of the embodiment of FIG. 12 of the present invention.

FIG. 14 is an oblique view of the embodiment of FIG. 12 of the present invention.

FIG. 15 is a lateral view of an embodiment of the deployer and dressing of the present invention.

FIG. 16 is an oblique view of the embodiment of FIG. 15 of the present invention.

FIG. 17 is a top view of the embodiment of FIG. 15 of the present invention.

FIG. 18 is a lateral view of an embodiment of a deployer and bandage of the present invention.

FIG. 19 is an oblique view of the embodiment of FIG. 18 of the present invention.

FIG. 20 is a lateral view of an embodiment of a deployer and bandage of the present invention.

FIG. 21 is an oblique view of the embodiment of FIG. 20 of the present invention.

FIG. 21a is a right oblique view of an embodiment of a deployer and bandage of the present invention.

FIG. 21b is a left oblique view of the embodiment of FIG. 21b of the present invention.

### DETAILED DESCRIPTION OF THE PREFFERED EMBODIMENTS

Before discussing the structure of the invention in detail, note that the layers of materials used in the structure are quite thin. In the various figures, the thicknesses are sometimes exaggerated for clarity of illustration. In particular, layers of adhesive are usually not shown, as the adhesive is generally coated directly onto the components and is not technically a separate part. All adhesive coated parts are clearly identified and the sides and portions of the components that are adhesive coated are clearly defined. Recognize, also, that when exaggerations occur, they also exaggerate the curvatures that occur in the drawings at the overlapping intersections of various layers.

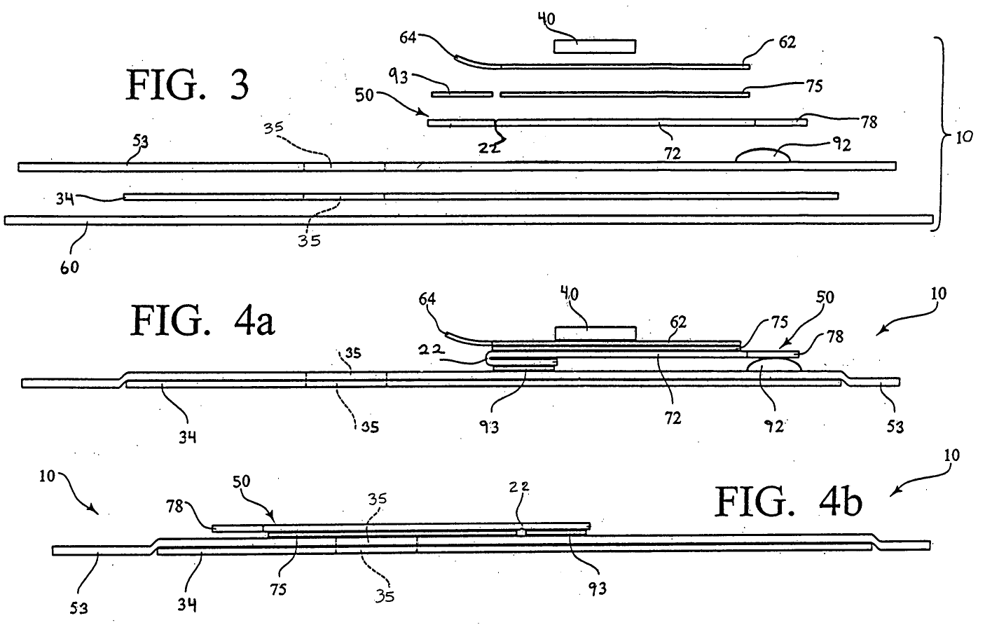

FIGS. 1-4 are drawings of one embodiment of the present invention. FIG. 1 shows an embodiment of the present invention from above as it may be packaged before use. The complete dressing is generally designated as 10. The dressing 10 is composed of multiple components layered on top of each other to form the finished product. FIG. 2 shows each of the individual components forming the complete dressing 10. FIG. 3 shows each of these components stacked, but separated from each other, from a side view. Referring to FIGS. 1-3, the embodiment of the present invention shown in these figures includes an absorbent layer 34 sandwiched between a carrier paper 60 and a patch base layer 53.

The carrier paper 60 acts as a backing for the entire dressing during packaging and shipping and is removed just prior to application of the dressing 10. The carrier paper 60 is designed to affix to and release from an adhesive layer and so is composed of a material having these properties, for example, a plastic or waxed paper. Since the patch base layer 53 is slightly larger than the underlying absorbent layer 34, the carrier paper 60 contacts the entire bottom surface of the absorbent layer 34 and a portion of the bottom surface of the patch base layer 53.

The absorbent layer 34 directly contacts the skin at the medical procedure site when the dressing 10 is applied to the patient. The absorbent layer 34 must also be able to wick away fluids from the procedure site. As such, the absorbent layer 34 is composed of a material that is compatible with extended contact with human or animal skin and readily absorbs fluids. For example, the absorbent layer 34 can be composed of cotton, or other similar natural absorbent fibers or absorbent polyurethane. One of skill in the art would recognize other suitable materials alone or in combination having the dual qualities of absorbency and compatibility with human skin contact that would work equally well within the scope of the present invention. The absorbent layer 34 defines a procedure site aperture 35 that passes completely through the absorbent layer 34. The dressing 10 is placed over the procedure site so that the procedure site aperture 35 completely surrounds the procedure site with enough open space available for the procedure to be performed. The absorbent layer 34 may further have an adhesive coating on its bottom skin-contacting surface (not shown) that releasably adheres the dressing 10 to the procedure site so that separate bandaging materials are not required. The thickness of the absorbent layer 34 can vary depending on the particular needs of the procedure. For example, if the procedure is expected to produce a lesion of considerable height, the absorbent layer 34 can have a thickness greater than what might ordinarily be used. A thicker absorbent layer 34 may also be desirable when the procedure results in excess fluids. As a non-limiting example, the absorbent layer 34 of the embodiment can vary as needed between about 1/16^{th} of an inch and about one (1) inch.

The patch base layer 53 is positioned on top of the absorbent layer 34 and can extend beyond the edges of the absorbent layer 34. The patch base layer 53 functions to mold the absorbent layer 34 to the contours of the skin and provide a waterproof barrier over the procedure site. It may also be desirable to use materials that are transparent. Therefore, the patch base layer 53 may be comprised of any material or materials, as is generally known in the art, that provides features that may include flexibility, non-toxicity, and transparency. For example, the patch base layer 53 can be made from any of a number of natural and synthetic polymers, including, but not limited to, rubber or polyurethane. The patch base layer 53 also defines a procedure site aperture 35 preferably of the same size and shape as the one formed through the absorbent layer 34. The procedure site aperture 35 should be located on the patch base 53 so that when the dressing 10 is assembled, the apertures 35 formed through the absorbent layer 34 and the patch base layer 53 line up so as to form an aperture through both these layers, providing a clear line of sight through the procedure site aperture 35 to the procedure site when the dressing 10 is applied. The patch base layer 53 may also have an adhesive coating on its bottom surface (not shown) both for the benefit of adhering the dressing 10 to the skin at the procedure site and for adhering the absorbent layer 34 to the patch base layer 53. Preferably, the adhesive layer on the patch base layer 53 is sufficient to adhere the dressing 10 to the patient's skin so that an adhesive layer is not also required on the absorbent layer 34.

An embodiment of the present invention may further include an observation door 50. When disassembled, as shown in FIG. 3, the observation door 50 can be a flat sheet having a flap member 72 and a fixed member 96 joined to each other at a hinge 22. When assembled as shown in FIGS. 1, 4a and 4b, the fixed member 96 is folded under the flap member 72 at the hinge 22 and adhered to the patch base layer 50 with, for example, a permanent adhesive 93. This conformation positions the observation door 50 in an open position, which is shown in FIGS. 1 and 4a. The observation door 50 can be held in its open position by an adhesive bead 92 located on the patch base layer 53. The adhesive bead 92 is a releasable adhesive so that the flap member 72 of the observation door 50 can be pulled free and swiveled on the hinge 22 to a closed position.

As shown in FIG. 4b, when the observation door 50 is placed in its closed position, the procedure site aperture 35 is covered and protected by the flap member 72 of the observation door 50. Since the clinician may wish to observe the site over time without always needing direct access to the site, the observation door 50 may be made from a transparent material. Alternatively, the flap member 72 of the observation door 50 may simply have a transparent window (not shown) through which the site can be observed, with the remainder of the observation door 50 constructed from an opaque material. Further, since the observation door 50 may also be closed for extended periods of time, it may be desirable to construct at least the portion of the flap member 72 covering the aperture 35 from a gas permeable material or provide small pores through the flap member 72. Suitable materials with one or more of the above listed properties would be obvious to one of skill in the art. As non-limiting examples, such materials may include, but are not limited to, silicone and polyurethane, or other natural or synthetic polymers with minute pores throughout, as can be accomplished with lasers or other fine puncturing tools.

A releasable adhesive 75 can cover the top surface (when open) of flap member 72 so that when the flap member 72 is positioned in its closed position, the releasable adhesive 75 will hold the observation door 50 in its closed position. However, because the releasable adhesive 75 is releasable, the observation door 50 can be opened after it is closed so that access can again be gained to the procedure site. The flap member 72 may have a positioning tab 78 that facilitates moving the observation door 50 between its open and closed positions. The positioning tab 78 can be placed on a side opposite the hinge 22, where it will likely be most effective.

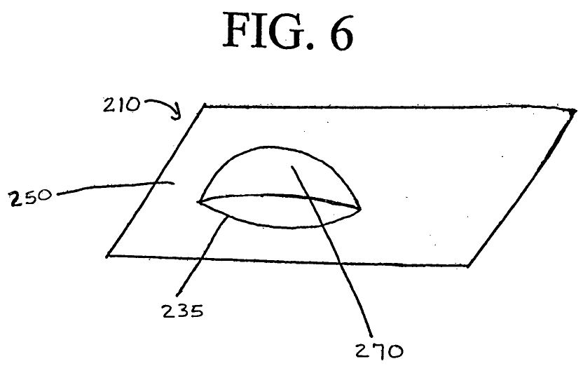

The observation door 50 may have a conformation other than flat. For example, it may have a convex or bubble shape, beneficial if a lesion produced by the procedure reaches a thickness large enough that the thickness of the absorbent layer 34 is insufficient to keep the lesion from directly contacting the observation 50. A convex shape to the observation door 50 could provide added height above the procedure site. Referring to FIG. 6, the dressing 10 could be initially applied until the lesion grew too large and then the entire dressing 10 is replaced with a simpler dressing 210 comprised of a flexible adhesive layer 250 similar to the patch base layer 53 defining an aperture 235 and surrounding an observation window 270 that is so steeply convex-shaped so as to form a dome over the lesion. The observation window 270 does not open to allow access to the site as does the observation door 50. The dressing 210 may or may not include an absorbent pad, depending on the particular need.

As a further embodiment of the present invention, the dressing 10 may include an absorbent pad 40. As shown in FIGS. 1 and 3, the absorbent pad 35 can be mounted to a backing 62, which in turn can be releasably affixed to the flap member 72 of the observation door 50. The releasable adhesive 75 can hold the absorbent pad 35 backing 62 to the observation door 50. The absorbent pad 40 can be made from any absorbent material, as would be generally known by one of skill in the art, with wicking properties and while also being compatible with contact with animal or human tissue. For example only, the absorbent pad 40 could be made from cotton or absorbent polyurethane. Further, the absorbent pad 40 can be constructed from the same material as the absorbent layer 34 or even cut from the absorbent layer 34. Cutting the absorbent pad 40 from the absorbent layer 34 provides the added advantages of both saving on materials costs and assuring that the absorbent pad 40 is exactly the same diameter as the absorbent layer 34, which then ensures a tight fit of the absorbent pad 40 within the absorbent layer 34. Even further, additional absorbent pads (not shown) separate form the dressing can be provided if follow up cleansing of the procedure site is required at any time during the monitoring process.

In practice, the absorbent pad 40 facilities safe sharps handling by the practitioner. After the procedure is performed, the practitioner can simply close the observation door 50, which brings the absorbent pad 40 in contact with the procedure site. If the absorbent pad 40 is sized to exactly fill the open space of the aperture 35, then no further pressure application or adhesives are required to hold the absorbent pad 40 in place. Thus, the clinician can perform the procedure and easily swab and cover the procedure site with one quick motion. This leaves the clinician free to properly focus on safely discarding any contaminated sharps used during the procedure without further attention to the procedure site. The absorbent pad 40 will wick away any fluids from the procedure site and cover the site while the clinician attends to other matters. Once all the excess fluid has been absorbed away from the site, the absorbent pad 40 is removed from the observation door 50 by pulling the backing 62 away from the observation door 50 and then closing the door 50. A removal tab 64 can be attached to the absorbent pad backing 62 to facilitate removal of the absorbent pad 40 and backing 62 from the door 50. In one embodiment, the removal tab 64 is extended and the absorbent pad 40 is situated on the lower side of the extension in such a way that the act of removing the absorbent pad 40 by pulling the removal tab 64 to the left (as seen in FIG. 3) would rotate the observation door 50 on its hinge 22 to close the observation door 50 over the procedure site aperture 35 with one single movement.

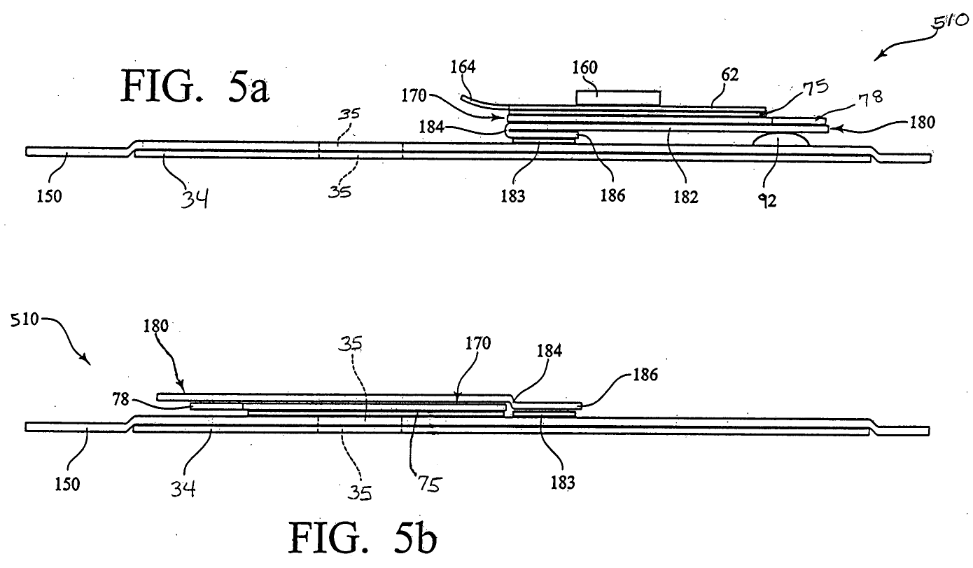

It may be desirable to have a dressing wherein the procedure site is not generally visible for aesthetic reasons. In another embodiment of the present invention, an opaque outer door 180 can be affixed to a dressing 510, as shown in FIGS. 5a and 5b. The outer door 180 is positioned so that it covers an observation door 170 when both doors are in a closed position, as shown in FIG. 5b. In FIGS. 5a and 5b, the outer door 180 is placed so that a fixed member 186 of the outer door 180 is adhered with a permanent sealant 183 to a patch base layer 150. A flap member 182 is then joined to the fixed member 186 by a hinge 184. The hinge 184 permits the outer door 180 to pivot between an open (FIG. 5a) and a closed (FIG. 5b) position. The observation door 170 is then affixed to the outer door 180. The exact placement of the outer door 180 is only relevant to the present invention in that when the outer door 180 is in its closed position, it covers a procedure site aperture 35 so as to prevent direct viewing of the aperture 35. For example, the observation door 170 and the outer door 180 can both be hingedly affixed to the patch base layer 150 and the outer door 180 is still capable of concealing the aperture 35 when closed. In another example, the outer door 180 can be made by simply cutting away all but one edge of the patch base layer 150 so that a flap is formed that becomes the outer door 180. The outer door 180 may also have a releasable adhesive on a surface so that the outer door 180 can be held in its closed position. If gas transfer to and from the procedure site is important, it may be desirable to either manufacture the outer door 180 from a gas permeable material, or apply the adhesive only to one edge of the outer door 180 so that gas can still transfer around the remaining open edges.

In another embodiment, the visible surface of the opaque outer door 180 when it is in its closed position can be decorated with colors or designs to provide a more pleasing outer appearance, since the patient may be required to wear the dressing 510 for extended time periods. For example, the outer door 180 can be stamped with children's cartoon characters, slogans or commercial logos. It would be obvious to one of skill in the art to provide an outer door 180 with appropriate designs and still remain within the scope of the present invention.

With the above embodiments of the present invention in mind, several non-limiting example procedures and a generalized method of using the embodiments are now provided. The dressing of the present invention is particularly useful for invasive procedures that require monitoring of the procedure site over time. For example, the dressing may be useful for minor surgical procedures such as suturing of lacerations or removal of small amounts of tissue. As another example, the dressing may be useful for certain invasive skin tests such as allergy testing or tuberculosis skin tests where the site must be repeatedly monitored for a reaction over time and yet needs to remain protected from the environment. Another example of a procedure in which the dressing of the present invention may be useful is for certain vaccinations that require periodic monitoring over time. For example, the current vaccination against Variola virus, the virus that causes smallpox in humans, is a live virus vaccine that must be closely monitored for development of a characteristic lesion. If no lesion develops, then it is likely the administered vaccine was ineffective at providing immunity against smallpox. However, in rare cases, the smallpox vaccine can stimulate a reaction that is excessive to the point of requiring further medical intervention. In either case, the vaccination site must be covered to protect the site from the environment (and conversely protect others from any infectious exudate produced at the site) and also regularly monitored. A dressing embodiment of the present invention satisfies both these requirements without the need for regular dressing changes. Additional uses for the dressings of the present invention will be evident to those of skill in the art.

In use, the clinician first cleans the site upon which the procedure is to be performed. Next, the dressing 10, 510 is affixed over the procedure site, for example by removing the carrier paper 60 and pasting to the skin. The medical procedure is then performed at the site through the procedure site aperture 35. The observation door 50, 170 is then closed and if an absorbent pad 40, 160 is present it is wedged into and enters the aperture 35. The absorbent layer 34 around the aperture 35 and the absorbent pad 40, 160 together absorb any tissue exudate and excess procedure fluids present at the site. Meanwhile, the clinician is free to focus attention on safely disposing of any contaminated sharps and other materials. The absorbent pad 40, 160 is then removed and the observation door 50, 170 is closed. If an opaque outer door 180 is present, it may also be closed. Progress at the site over time is monitored by lifting the outer door 180, if present, to its open position and viewing the site through the observation door 50, 170. If access to the site is required (such as to absorb more exudate or otherwise clean the wound with an additional absorbent pad) the observation door 50, 170 can simply be raised to its open position and then closed again when the procedure is finished. While the patient is wearing the dressing 10, 510, the site is protected from the environment. When the site has healed, the dressing 10, 510 can be removed. Thus, the patient does not have to be bothered with the discomfort and expense of continuously changing dressings over the course of procedure site monitoring and healing.

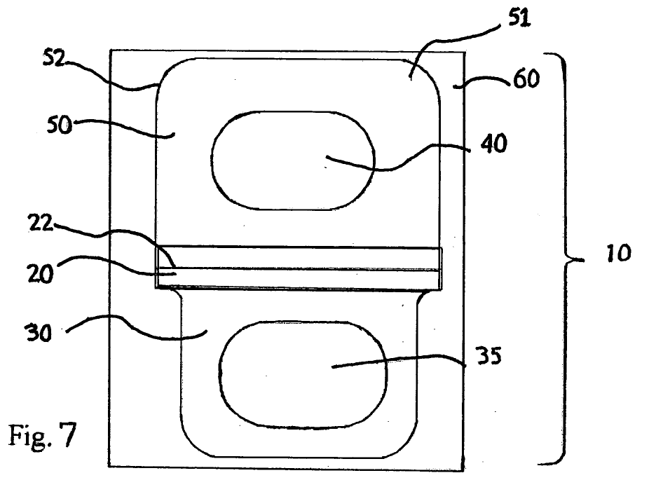

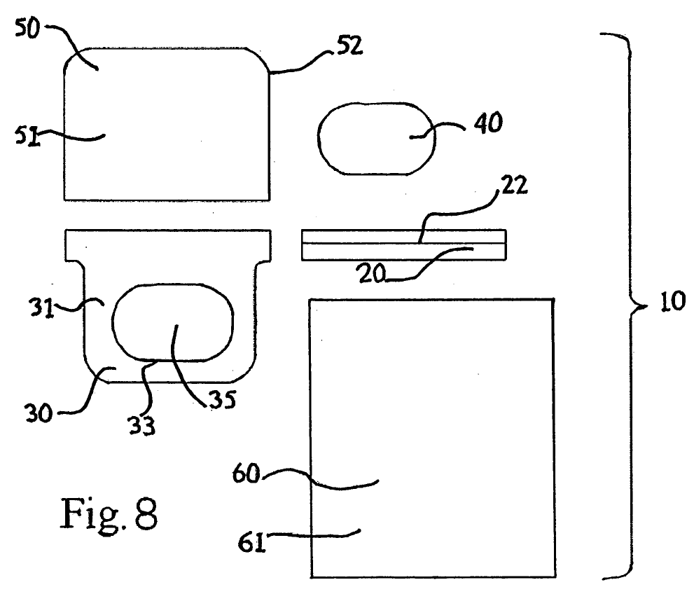

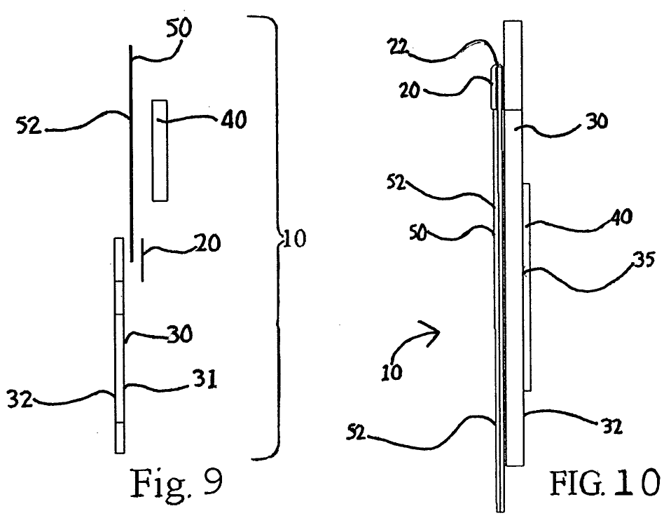

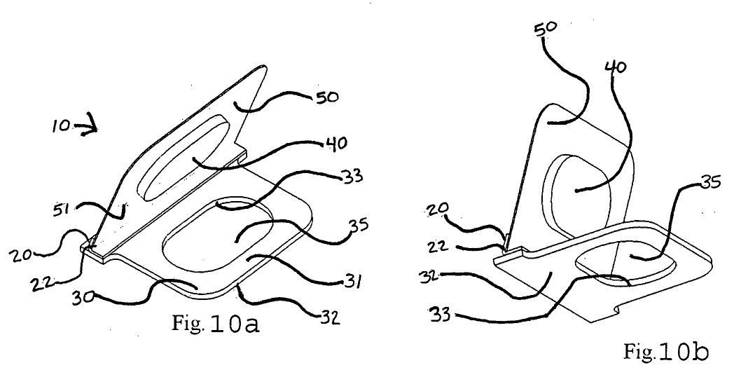

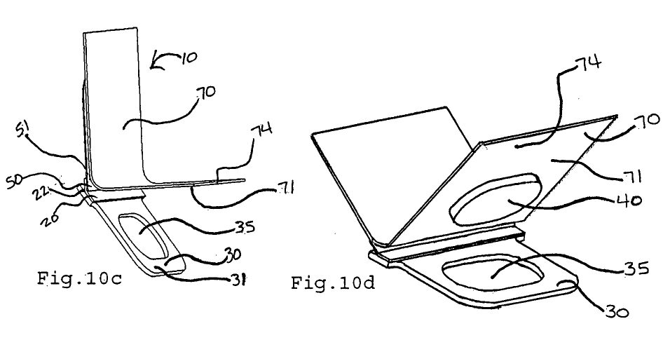

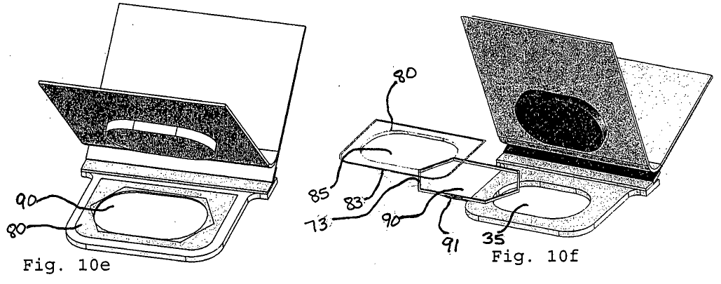

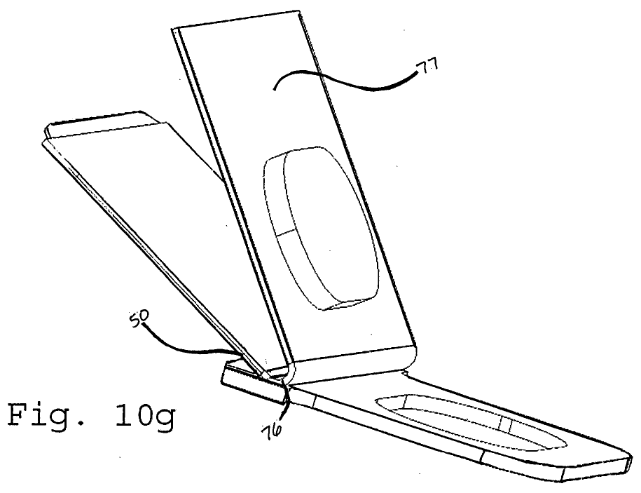

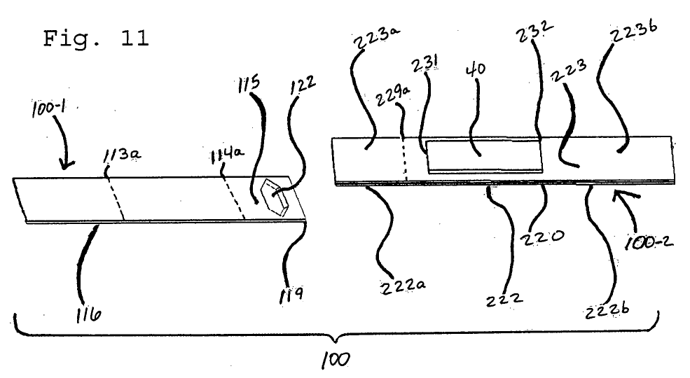

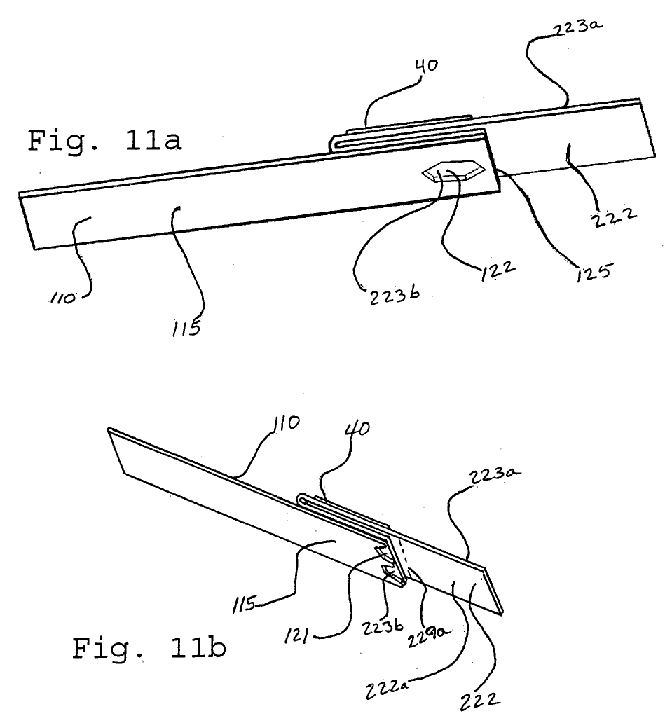

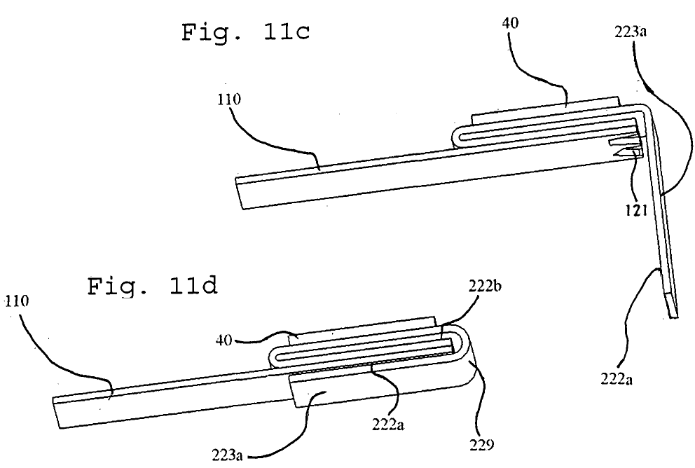

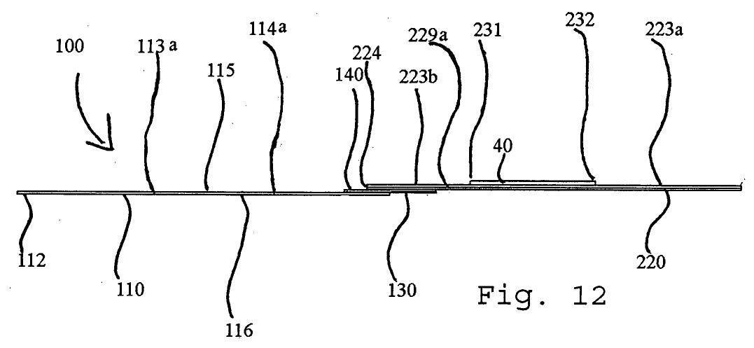

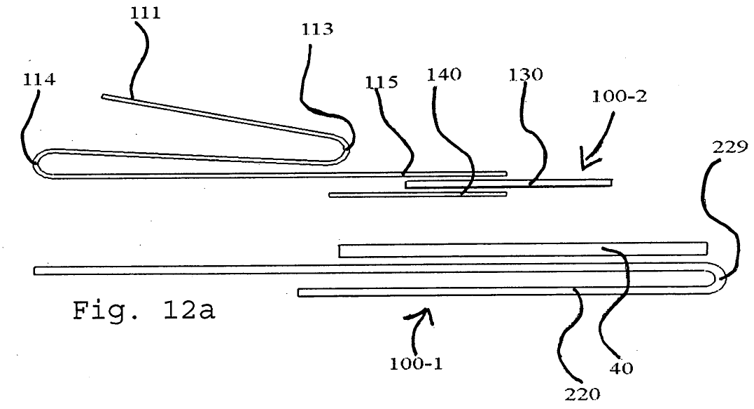

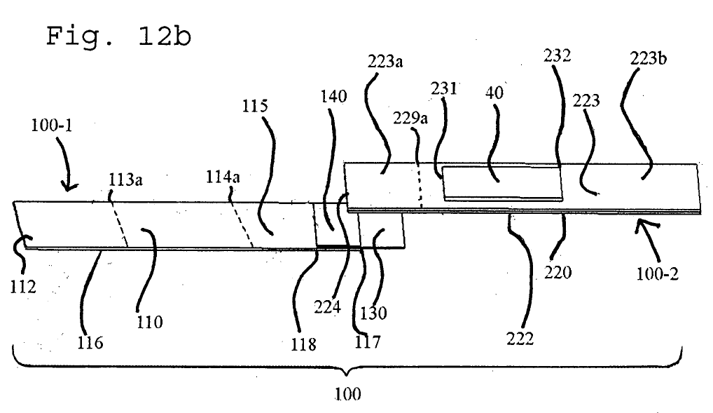

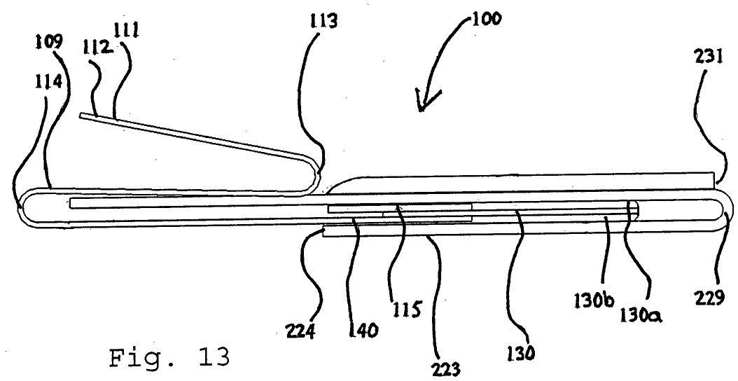

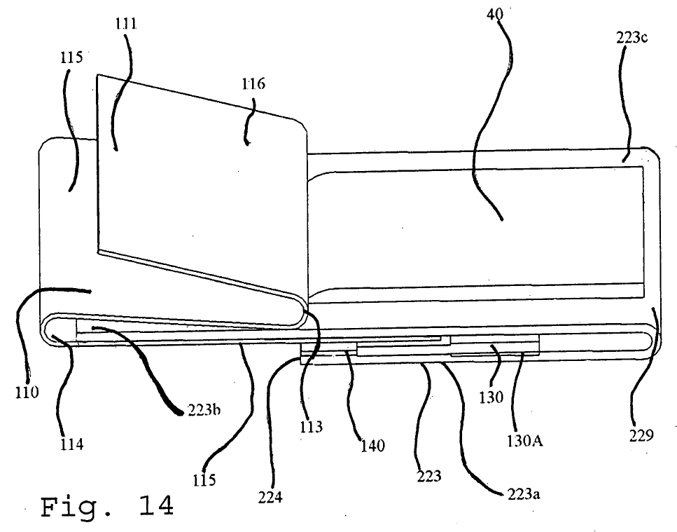

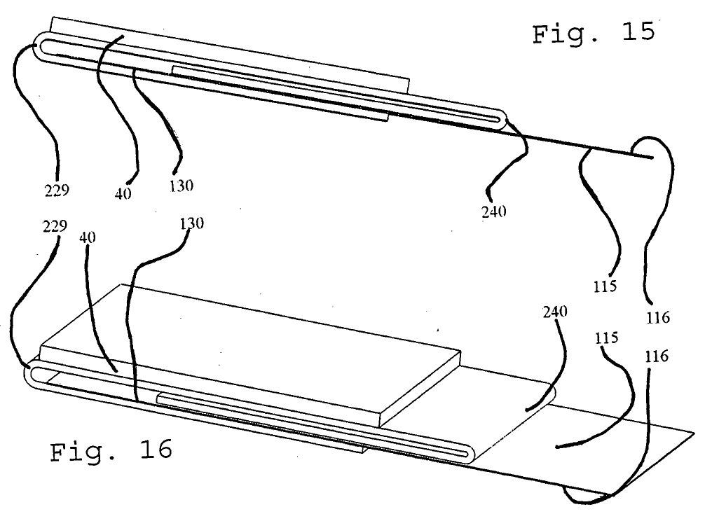

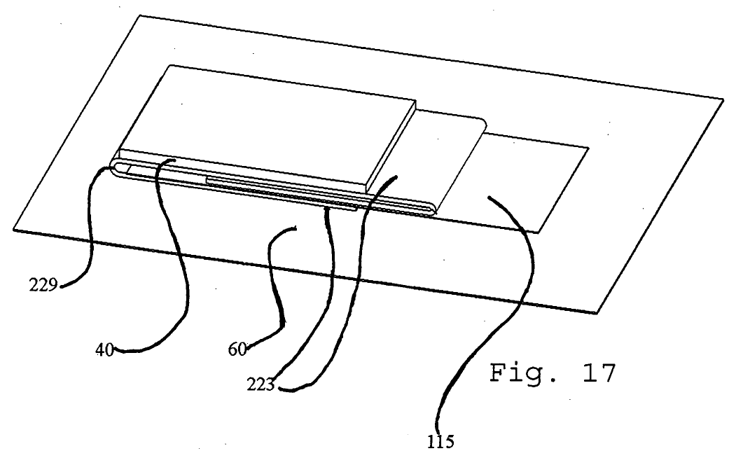

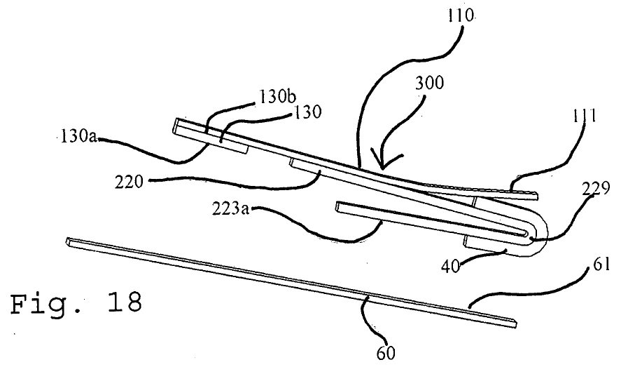

FIGS. 7 through 10b depict an embodiment of the present invention. Referring to FIGS. 7 and 8, the embodiment of the dressing 10 of the present invention is shown from above as it is open and may be packaged prior to use. The dressing 10 is composed of multiple components layered on top of each other to form the finished product. Referring to FIGS. 9 and 10, the components of the dressing 10 are stacked from a side view. The dressing 10 has an optional carrier paper 60 coated with a release coating 61, such as, for example only, silicone. The dressing 10 includes a door 50 with a back surface 52 removably adhered to the carrier paper 60 with the release coating 61 on the carrier paper 60. On the opposing side of the back surface 52 is the front surface 51, upon which is centrally and adhesively positioned an absorbent pad 40. A hinge 22 and a flexible material 20 connect the door 50 to the bandage body 30. The base 31 of the bandage body 30 surrounds a lesion, wound, procedure site, etc. The bandage body 30 has an aperture 35 formed by a ring 33 to surround the lesion, wound, procedure site, etc. The bandage body 30 defines the procedure site aperture 35 that passes completely through the bandage body 30. The dressing 10 is placed over the procedure site so that the procedure site aperture 35 completely surrounds the procedure site with enough open space available for the procedure to be performed.

The bandage body adhesive 32 on the bandage body 30 is removably adhered to the carrier paper 60 by the release coating 61 on the carrier paper 60. The carrier paper 60 acts as a backing for the entire dressing 10 during packaging and shipping, and is removed just prior to application of the dressing 10. The carrier paper 60 is designed to affix to and release from an adhesive layer and so is composed of a material having these properties, such as, for example only, a plastic or waxed paper. When the bandage body 30 is removed from the carrier paper 60, the bandage body adhesive 32 is removably adhered to the skin of a subject.

The absorbent pad 40, when the door 50 is rotated about the hinge 22 toward the bandage body 30 to close, may fit into the aperture 35 when completely closed. The absorbent pad 40 may directly contact the skin at the medical procedure site when the dressing 10 is deployed, that is, when the door is completely closed. Referring to FIG. 10, the dressing 10 in the deployed position is shown. In this embodiment, the absorbent pad 40 slightly protrudes through the aperture 35 once deployed and contacts the skin. In some embodiments, the absorbent pad 40 does not encroach upon the aperture 35 when the invention is deployed. In all embodiments, the absorbent pad 40 should be able to wick away fluids from the procedure site. The absorbent pad 40 may be composed of a material that is compatible with extended contact with human or animal skin and readily absorbs fluids, such as, for example only, cotton or other similar natural absorbent fibers or absorbent polyurethane. One of skill in the art would recognize other suitable materials, alone or in combination, having the dual qualities of absorbency and compatibility with human or animal skin contact that would work equally well within the scope of the present invention.

The thickness of the absorbent pad 40 can vary depending on the particular needs of the procedure. For example, if the procedure is expected to produce a lesion that produces copious fluids, the absorbent pad 40 can have a thickness greater than what might be ordinarily used. As a non-limiting example, the thickness of the absorbent pad 40 of one embodiment can vary as needed between about 1/32^{nd} of an inch and about one (1) inch and may be partially cut-away to create room for an elevated lesion.

Referring now to FIGS. 10a and 10b, the dressing 10 is shown in the partially deployed, or closed, position, with the door 50 rotated about the hinge 22. The bandage body adhesive 32 molds the bandage body 30 to the contours of the skin and provides a waterproof barrier around the aperture 35 and over the procedure site. Transparent materials may be desirable. Therefore, the bandage body 30 may be comprised of any material or materials, as is generally known in the art that provides features that may include flexibility, non-toxicity, and transparency. For example only, the bandage body 30 can be made from any of a number of natural and synthetic polymers, including, but not limited to, rubber or polyurethane. The procedure site aperture 35 in the bandage body 30 may preferably, but not necessarily, be of the same shape and slightly larger size as the absorbent pad 40. In this embodiment, the procedure site aperture 35 is located on the bandage body 30 in such a position so that the aperture 35 formed through the bandage body 30 and the absorbent pad 40 line up such that the absorbent pad 40 fits cleanly and snuggly inside the aperture 35 and makes contact with the procedure site when the dressing 10 is applied and the door 50 is closed.

When the dressing 10 is deployed, the procedure site aperture 35 is covered and protected by the door 50. Furthermore, and referring to FIGS. 10c and 10d, since the clinician may wish to observe the procedure site over time without always needing direct access to the site, the door 50 may be made from a transparent material, and the absorbent pad 40 may be removable. In this embodiment, the absorbent pad 40 is positioned on a piece of release-paper backing 70 coated on a release tab 74. The absorbent pad 40 is attached to the release-paper backing 70 with a releasable coating 71. The release-paper backing 70 is, in turn, releasably affixed to the front surface 51 of the observation door with the releasable coating 71. The combined component made of the absorbent pad 40 adhered to the release-paper backing 70 can be used to deploy the dressing 10 and then can be removed and discarded after the dressing 10 is deployed. Again, the absorbent pad 40 can be made from any absorbent material, as would be generally known by one of skill in the art, with wicking properties and compatible with animal or human skin contact, such as, for example only, cotton or absorbent polyurethane.

The observation door 50 may simply have a transparent window through which the site can be observed, with the remainder of the observation door 50 constructed from an opaque material. Additionally, observation and treatment of the site may be further aided by using a non-permanent and re-positionable adhesive on the front surface 51 of the door 50. This configuration would permit the door 50 to be opened as needed to access the site for care or observation. Further, the door 50 may have a conformation other than flat, such as a convex or bubble shape. A convex shape may be beneficial, for example, if a lesion produced by the procedure reaches a thickness large enough that the thickness of the bandage body 30 is insufficient to keep the lesion from directly contacting the door 50. A convex or bubble shape to the door 50 would provide added height above the procedure site.

Further, since the observation door 50 may also be closed for extended periods of time, it may be desirable to construct at least a portion of the door 50 covering the aperture 35 from a gas permeable material or provide small pores through the door 50. As non-limiting examples, such materials with the above-listed properties may include silicone, polyurethane, or other natural or synthetic polymers with minute pores throughout, as can be accomplished with lasers or other fine puncturing tools. The door 50 may be made of a solid material or it may have holes, vents, or pores in it to allow the site to breathe and help prevent moisture buildup. The vented door 50 may or may not be covered by a semi-permeable membrane that would prevent micro-organisms from passing either way through the door, but would allow moisture to exit the procedure site to help keep the humidity from building up under the door.

In practice, the absorbent pad 40 facilitates safe sharps handling by the practitioner. After the procedure is performed, the practitioner can simply close the door 50, which brings the absorbent pad 40 close to or in contact with the procedure site. If the absorbent pad 40 is sized to exactly fill the open space of the aperture 35, then no further pressure application or adhesives is required to hold the absorbent pad 40 in place. Thus, the clinician can perform the procedure and then easily swab and cover the procedure site with one quick motion. Additionally, the absorbent pad 40, whether the same size as the aperture 35 or larger, can be pushed against the needle barrel and the skin as the needle barrel is being removed from the tissue after a needle puncture has been performed. In this position, the absorbent pad 40 and door 50 can be used to shield the clinician from the blood splatter or aerosol which could occur from the puncture or from the triggering of a retractable type safety needle. Once the needle clears the aperture 35, the door 50 can be fully closed so that the front surface 51 of the door 50 contacts the base 31 of the bandage body 30 and seals the site closed. This allows the clinician to focus on safely discarding any contaminated sharps used during the procedure without further attention to the procedure site. The absorbent pad 40 will wick away any fluids from the procedure site and cover the site while the clinician attends to other matters. The material of the bandage body 30 forms an adhesive 360° ring 33 around the procedure site. Once the door 50 is closed, and the dressing 10 is thus in the deployed position, the front surface 51 of the door 50 adheres the door 50 to the base 31 of the bandage body 30, and a complete seal is formed over the procedure site.

Referring now to FIGS. 10c and 10d, the absorbent pad 40 is adhered to the removable release tab 74, which can be completely removed from the dressing. Once the excess fluid has been absorbed away from the site, the absorbent pad 40 can be removed from the observation door 50 by pulling the release-paper backing 70 away from the observation door 50 and then closing the door 50. The absorbent pad 40 may be adhesively attached to a graspable release tab 74 to facilitate removal of the absorbent pad 40 and release-paper backing 70 together from the door 50. The release tab 74 is extended and the absorbent pad 40 is situated on the lower side in such a way that the release-paper backing 70 can be rotated towards the bandage body 30 and the absorbent pad 40 still attached to the paper release-paper backing 70 can be placed against a needle shaft or puncture site and then the door 50 can be re-opened. Next, pulling the release tab 74 away from the door 50 would rotate the door 50 on its hinge 22 to close the door 50 over the procedure site aperture 35 with one single movement and the release-paper backing 70 and the absorbent pad 40 are removed together from the front surface 51 of the door 50. The release-paper backing 70 with the door 50 can then be discarded as a single piece.

As an example of use, the clinician first cleans the site upon which the procedure is to be performed. Next, the dressing 10 is affixed over the procedure site by removing the carrier paper 60 and affixing the patch to the skin by pressing the bandage body adhesive 32 against the skin. The medical procedure is then performed at the site through the procedure site aperture 35. The door 50 is then closed and if an absorbent pad 40 is present, it enters and is wedged into the aperture 35. The absorbent pad 40 absorbs any blood, tissue exudate, and excess procedure fluids present at the site. Meanwhile, the clinician is free to focus her attention on safely disposing of any contaminated sharps.

Referring now to FIGS. 10e and 10f, two layers have been added to add a level of safety to the invention. Only the numeric designations that refer to the two new layers are shown in these drawings. One of the additional layers is an injection material 90, which is a clear or translucent non-coring, flexible, self-sealing, non-pyrogenic material such as certain formulations of thermoplastic elastomeric film. The injection material 90 completely covers the aperture 35 and can be held in place over the aperture 35 by a ring of film adhesive 91 between the underside periphery of the aperture 35 and the base 31 of the bandage body 30. An additional layer of material, a retaining layer 80, can be layered over the injection material 90 to hold the injection material 90 in place. The retaining layer 80 can have the necessary qualities of the injection material described above or it 80 can have a retaining layer aperture 85 to expose and create unimpaired access to the injection material 90. The adhesively coated retaining layer bottom 83 adheres to the top surface of the injection material 90 at the periphery 73 and to the base 31 of the bandage body 30, and thus holds the injection material 90 firmly in place on the bandage body 30. The retaining layer 80 can be transparent, translucent, or opaque.

The retaining layer 80 and the injection material 90 provide an additional layer of protection between the procedure site and the healthcare provider. An injection can be performed by penetrating the injection material 90 with a hypodermic needle on a syringe. Most or all blood or body fluid loss that may occur beneath the injection material 90 will remain on the patient's side of the injection material 90 even after the needle is removed because the hole created by the needle closes and seals itself as the needle is removed due to the properties of the injection material 90. In the event of bio-hazardous materials passing through the injection material 90 and staying on the surface of the injection material 90, closing the door 50 will isolate the bio-hazardous material by sealing it under the door 50, which is generally made to include a layer which is impervious to the typical biohazards of bacteria and viruses.

Referring now to FIG. 10g, there are clearly other ways to hold the door 50 open and to attach the door 50 to the base 31 without changing the invention to observe the procedure site through the aperture 35. For example, instead of a separate hinge 22, one edge of the door 50 may be folded under in such a way that the adhesive of the door 50 contacts the base 31 and the release-paper backing 70 holds the door in place by an overlapping fold 76 which blocks the hinge 22 from rotating but still allows the front section of the release paper 77 to rotate and bring the absorbent pad 40 down to the aperture 35. Also, the adhesive that holds the door 50 and the base 31 together when the dressing 10 is deployed (i.e., closed) may be on the base 31, rather than on the door 50. Furthermore, the bandage body 30 may be completely absent and the flexible material 20 near the hinge 22 may be adhered directly to the skin. If the door 50 is too heavy, a bit of adhesive between the back surface 52 of the door 50 and the skin, to hold the dressing 10 in its un-deployed configuration, may be necessary.

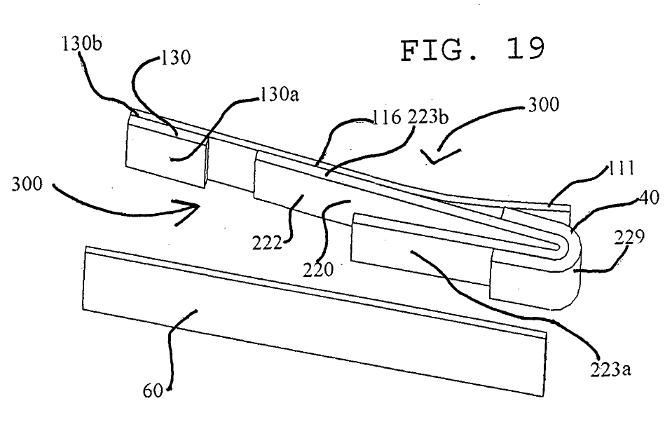

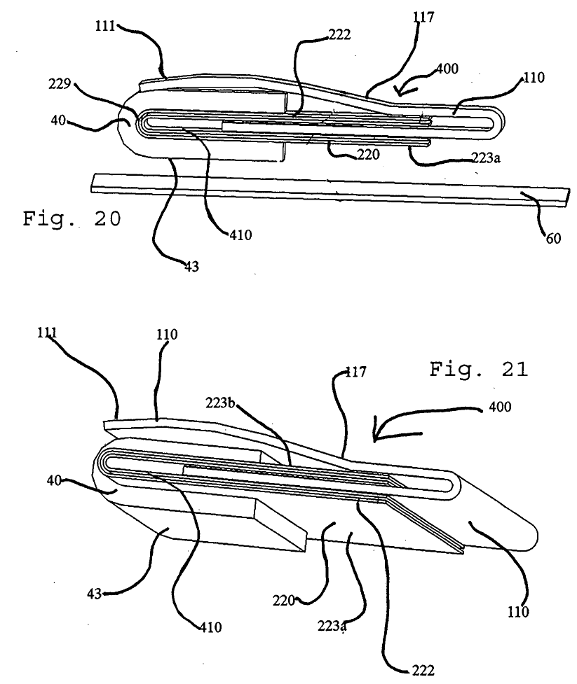

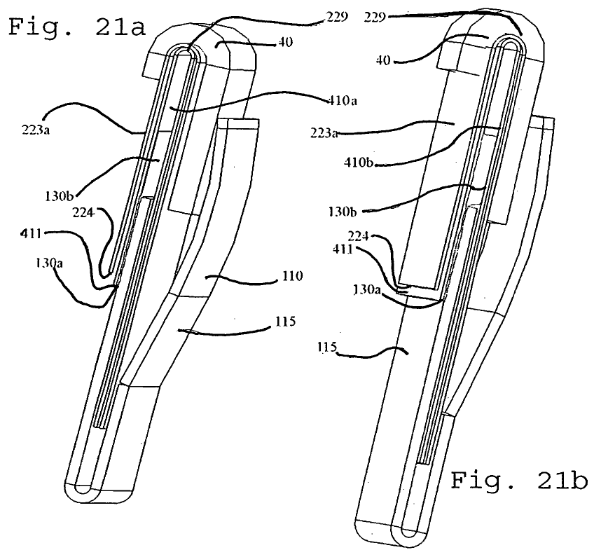

Referring next to FIGS. 11, 11a, 11b, 11c and 11d, a deployer 100 is presented having a deployer aperture 122 placed at the end of the flexible release-paper 110. The versions of the device shown here demonstrates one more of the nearly unlimited conformations of the deployers 100. In this embodiment, the deployer component 100-1 consists only of the flexible release-paper 110 with some strategically placed deployer apertures 121, 122 in, at, or near one end 119 of the flexible release-paper 110. When the device is folded, as in FIGS. 11c and 11d, the absence of material between the adhesive flap 223b and the bandage back flap 222a at the deployer apertures 121, 122 allows the adhesive flap 223b to contact and adhere to the bandage back flap 222a. Since the adhesive flap 223b extends through the deployer apertures 121, 122, it will the adhere adhesive flap 223b to the non-adhesive surface of the bandage back flap 222a when folded at a potential hinge location 229a (see also FIG. 12) to create the deployer hinge 229 and to bring the adhesive flap 223b into contact with the bandage back flap 222a through the deployer apertures 121, 122. Note that the flexible release-paper 110 and the bandaging material 220 are very thin and flexible; therefore there is little to prevent the adhesive flap 223b from contacting the bandage back flap 222a through the deployer apertures 121, 122. Also, note that the deployer aperture 121 through flexible release-paper 110 of FIG. 11b are different sizes, shapes, and locations than the deployer aperture 122 of FIG. 11a, demonstrating how different structural embodiments of this invention are still the same invention. Both the embodiments of FIG. 11a and FIG. 11b are folded exactly like embodiments of FIGS. 15-17, see below, as is demonstrated by FIGS. 11c and 11d.

FIG. 11b shows the embodiment of FIGS. 11 and 11a with wedges removed from the end 119 of the flexible release-paper 110 to form the deployer apertures 121. The invention with this embodiment functions exactly as does the that of FIG. 11a, demonstrating that various configurations that permit the formation of the deployer hinge 229 by adhesively attaching the bandage back flap 222a to the adhesive flap 223b are different embodiments of the same invention.

The bandage component 100-2 of the deployer 100 is comprised of a bandaging material 220, such as a cloth or polyurethane material with a bandage adhesive coating 223 on one side and a cloth, plastic, polyurethane, or other non-adhesive bandaging material on the non-adhesive back 222. Many different materials and shapes and sizes of bandages could conform to the specifications and requirements of this component. One skilled in the art could easily substitute one bandaging material for another to construct another embodiment of the invention. The absorbent pad 40 is adhered to the bandage adhesive coating 223 at the adhesive flap 223b. In this embodiment, the bandaging material 220 has a very thin polyurethane layer laminated on the non-adhesive back 222.

In the embodiment shown in FIGS. 11 through 14, the bandage body 30 shown previously in FIGS. 7 through 10g is absent. FIGS. 12, 12a and 12b, show an embodiment of the deployer 100 prior to being folded into the useable configuration shown in FIGS. 13 and 14. FIG. 12 shows the deployer component 100-1 and the bandage component 100-2 in contact with one another. Folding the flexible release-paper 110 twice and the bandaging material 220 once completes this embodiment of the invention. In FIG. 12b, the two assemblies, the deployer component 100-1 and the bandage component 100-2, which are combined to form the deployer 100, are shown. The deployer 100 is comprised of a flexible release-paper 110, such as, for example only, paper coated with silicone on the deployer component bottom 116 and uncoated on the deployer component top 115. Also, the deployer 100 is comprised of a small piece of that same coated-material 140 and a piece of adhesive tape 130, coated on both sides with adhesive, which holds the invention in its closed configuration. The adhesive tape 130 is sandwiched between the non-coated sides of the two pieces of coated material 140 and flexible release-paper 110 at one end of the flexible release-paper 110. The combination of these three components forms the deployer component 100-1 for the deployer 100 of the embodiment of FIG. 12. Referring to FIG. 12b, in the construction of the deployer component 100-1, as an alternative to the adhesive tape 130, the end of the flexible release-paper 110, from left edge 118 to right edge 117, can be coated on one or both sides with a re-positionable adhesive or can have deployer apertures 121, 122 as seen in FIGS. 11a and 11b.

FIG. 12b shows how the deployer component 100-1 and the bandage component 100-2 are aligned prior to their being adhered together. The potential hinge location 229a where the bandage component 100-2 will be folded is shown as a dotted line. In FIGS. 12 and 12b, the potential left bend 113a and the potential right bend 114a will be locations of the left bend 113 and the right bend 114, respectively, as shown in FIG. 12a of the deployer component 100-1. The location and number of bends in general are almost arbitrary and there are many different location and numbers of bends in the flexible release-paper 110 that would give similar results and create the same invention.

To deploy the deployer 100, the flexible release-paper 110 is bent to about 170-degrees in the down direction, as shown in FIGS. 12 and 12b, at the potential left bend 113a and then bending the flexible release-paper 110 back about 189-degrees at the potential right bend 114a to produce the left bend 113 and the right bend 114, respectively, as shown in FIGS. 12a, 13, and 14. The adhesive tape 130 will be positioned in between the bandage back flap 222a and the bandage front flap 222b on the non-adhesive back 222 of the bandaging material 220. Adhesively apposing bandage back flap 222a and bandage front flap 222b holds them together and creates the deployer hinge 229. The chosen shape of the final flexible release-paper 110 is one of many choices for the bend locations and configurations and are shown as non-limiting examples. Referring now to FIG. 13, the exposed end 112 of the bandage component 100-2 then becomes a pull-tab 111, which is used to deploy the device by pulling it in the direction of left bend 113 and then pulling it back in the direction of the right bend 114 to rotate the bandaging material 220 around the deployer hinge 229. This motion straightens out the bandage against the skin or can be used to bring the absorbent pad 40 to the skin surface. Once the pull-tab 111 is pulled in the direction of the right bend 114, there is shear created between the adhesive on the adhesive tape 130, where it is adhered to the bandage back flap 222a, and the release-paper end 125. The release-paper end 125 is inside of the space between the bandage back flap 222a and the bandage front flap 222b. The adhesive does not go all the way to the lower edge 224 of the non-adhesive back 222 of the bandaging material 220. Hence, when the pull-tab 111 is pulled, some of the adhesive on the skin side adhesive surface 223a remains adhered to the skin before the shear begins, so the adhesive tape bottom 130b on the lower edge 224 of the flexible release-paper 110 can pull free from the non-adhesive back 222 of the bandaging material 220 without pulling the skin side adhesive surface 223a away or off of from the skin. The flexible release-paper 110 remains adhered to the non-adhesive back 222, while the bandaging material 220 is being rotated around the deployer hinge 229. Until the bandaging material 220 is rotated around the release-paper end 125, the flexible release-paper 110 remains adhered to the surface of the bandaging material 220 by some part of the adhesive tape 130, depending on the adhesive qualities of the adhesive tape 130. Further pulling the pull-tab 111 will free the deployer component 100-1 to be discarded, and will leave the bandage component 100-2 with the bandage adhesive 223 and absorbent pad 40 against the skin.

Folding the invention into the shape shown in FIGS. 12a, 13, and FIG. 14 completes the device. Folding the bandage component 100-2 at the potential hinge location 229a brings the non-adhesive bandage back flap 222a and the non-adhesive bandage front flap 222b to face one another to be held together by the adhesive tape 130 of the flexible release-paper 110. The non-adhesive side of the bandage is held in place by the adhesive components of the adhesive tape 130, which locks the embodiment, until deployed, in the unused and hinged position. There are numerous methods of holding the deployer hinge 229 locked in this open position near the procedure site.

With the above embodiments of the present invention in mind, several non-limiting example procedures and a generalized method of using the embodiments are herein provided. To deploy the deployer 100 during an injection procedure as an example, the deployer is positioned on the skin with the deployer hinge 229 close to where the injection is to be performed, about half the distance from absorbent pad left edge 231 and absorbent pad right edge 232. Hence, assuming a non-limiting 1 inch length, from the absorbent pad left edge 231 to the absorbent pad right edge 232, the deployer hinge 229 will be about ½ inch from the proposed procedure site.

Once a needle is inserted, either the pull-tab 111 or the release 109 at the right bend 114 is pulled in the direction of the deployer hinge 229. This movement pulls one side of the adhesive tape bottom 130b off of the bandage back flap 222a of the body of the bandaging material 220 and rotates the absorbent pad 40 around deployer hinge 229. The absorbent pad 40 is rotated all the way to the skin where the needle is inserted. The absorbent pad 40 is pressed against the needle barrel and the skin. As the needle is removed from the puncture, pressure with the pad 40 on the puncture site is maintained. The needle retraction is performed with the absorbent pad 40 and the bandaging material 220 shielding the needle to reduce the chance that any spray or aerosol from the procedure site could reach the clinician. Once the needle and the syringe fully clear the operative field, the adhesive flap 223b is pressed against the skin to complete the bandaging process. Then the pull-tab 111 is pulled towards the end of the bandaging material 220, the deployer component 100-1 is pulled away from the non-adhesive back 222 and the adhesive tape 130 is pulled off of the body of the non-adhesive back 222. The clinician then sets the adhesive by pressing the bandage against the skin from one end to the other. The deployer component 100-1 is then discarded and the procedure is complete.

The dressing of the present invention is particularly useful for invasive procedures that could lead to infections or needlestick injuries to second parties. For example, the dressing may be useful for minor surgical procedures such as suturing of lacerations or removal of small amounts of tissue. As another example, the dressing may be useful for certain invasive skin tests, such as allergy testing or tuberculosis skin tests, where the site must be repeatedly monitored for a reaction over time and yet needs to remain protected from the environment. Another example of a procedure in which the dressing of the present invention may be useful is for certain vaccinations. For example, the current vaccination against Variola virus, the virus that causes smallpox in humans, is a live virus vaccine. That means that every needle is not only assuredly contaminated with a somewhat virulent live virus, many of them may be contaminated with other very virulent microorganisms. A dressing embodiment of the present invention decreases the time that a clinician would be exposed to contaminated needles and hence would increase the safety of the procedure. Additional uses for the dressings of the present invention will be evident to those of skill in the art.

Referring now to FIGS. 15, 16, and 17, this embodiment varies from the previous embodiment in the way the bandage and the flexible release-paper 110 are folded, and, as in another embodiment described fully herein, the flexible release-paper 110 has adhesive only on the non-coated side of the end of the paper. Adhesive tape top 130A, as shown in FIG. 13, is absent from this embodiment. FIG. 17 is shown on a carrier paper 60 as it might be placed before being packaged.

FIGS. 18 and 19 show another embodiment of the deployer 300. It uses the same components as the embodiment of FIG. 12, but they are combined for use in a different way. The coated surface of the deployer component bottom 116 of the deployer component 110-1 faces the adhesive flap 223b of the bandaging material 220. The end of the flexible release-paper 110 most distant from the deployer hinge 229 has an adhesive coating bottom 130a on the adhesive tape 130 on the coated side of the deployer component bottom 116 of the flexible release-paper 110. The absorbent pad 40 is located around the deployer hinge 229 and is bent with the deployer hinge 229 in such a way that the deployer 300 can be lifted away from the carrier paper 60 without touching the bandage adhesive 223 and the adhesive coating bottom 130a, and the skin side adhesive surface 223a can be pressed and adhered against the skin to hold the device 300 in place while the procedure is being performed. The device deploys by lifting the pull-tab 111 to pull the flexible release-paper 110 coated release-surface of the deployer component bottom 116 away from the adhesive flap 223b of bandaging material 220. When this pull-tab 111 pulls the flexible release-paper 110 free of the bandaging material 220, the bandage is free to rotate on the deployer hinge 229, bringing the adhesive flap 223b in contact with the skin and the surface of the absorbent pad 40 into contact to cover the procedure site. With the skin side adhesive surface 223a and the adhesive flap 223b holding the absorbent pad 40 against the site, the bandaging material 220 is fully adhered in place over the procedure site. Pulling the pull-tab 111 such that the adhesive tape 130 is pulled off of the skin frees the deployer component 100-1 to be discarded and completes the procedure. Note, in this embodiment as shown, the absorbent pad 40 traverses the entire width of the bandaging material 220. An absorbent pad 40 not as wide as the bandaging material 220 would have bandage adhesive 223 borders all the way around its periphery and hence, when the bandage was deployed, the bandage adhesive 223 would form a seal around the absorbent pad 40 to fully isolate any potential infectious material under the bandage and within the sealed area.

Referring now to FIGS. 20 and 21, an embodiment of a deployer 400 is shown which uses a weakly adhered backing 410 as the non-adhesive back 222 of the bandaging material 200. This embodiment might be used to preemptively deliver a very thin bandaging material 220, such as a 1-mil polyurethane, to the skin without damaging the bandage. Here, the absorbent pad, the door, the hinge and the adhesive bandage combine to form one integral component without defining an aperture. This embodiment takes advantage of commercially available raw bandaging materials which have release layers of the weakly-adhered backing 410 laminated to the non-adhesive back 222 of the bandaging material 220. The procedure site is inferred from the location of the deployer hinge 229 and the location of the absorbent pad 40 and the deployer hinge 229 is retained in its open position and prevented from obscuring the operative field by a component which is either a weak adhesive or a mechanical restraint on the bandage, positioned so as to hold the device in its open configuration and so to define the deployer hinge 229 and to maintain the deployer hinge 229 in its open position until the device is deployed. The adhesive may be of a type that is non-permanent and is repositionable. Using an adhesive of this type, the door could be deployed and then put back in its hinged position several times during the course of a procedure. Here, the embodiment has a more permanent type of adhesive, two pieces of double sided tape or a coating of adhesive on the flexible release-paper 110 where the adhesive tapes 130. This adhesive tape 130 attaches the flexible release-paper 110 to a layer of weakly-adhered backing 410 which is weakly bonded to the non-adhesive back 222 of the deployer component bottom 116 of the bandaging material 220.

A non-limiting example of a material that can be used to make this deployer 400 is a clear thin polyurethane bandaging material 220 with a loosely bonded layer of polyethylene weakly-adhered backing 410 on the non-adhesive back 222 of the bandaging material 220. As a clarifying example of materials that might be used for this embodiment, 3M and Avery Dennison both manufacture materials of this nature, which are specifically designed to have a polyethylene for the weakly-adhered backing 410 removed from a thin film of polyurethane after the film adheres to the skin. At the time of this writing, the 3M material is experimental, is labeled MSX-6010 and is not commercially available, and the Avery Dennison material is labeled MED5030 and is commercially available. This embodiment is similar to an embodiment that would include the weakly bonded adhesive tape 130, which holds open the embodiment of FIGS. 11-14, except that it can be used to deliver very thin bandaging materials, such as 0.7 mil polyurethane, which can not be easily handled by themselves because they are very thin and adhesive coated on one side. Materials such as thin polyurethane films are often less than 1 mil thick.

Before deployment, the deployer 400 has the absorbent pad 40 bent at about 180 degrees in such a way that it can be grasped with two fingers, one on the flexible release-paper 110 at the pull-tab 111 and the other between the absorbent pad 40 and the carrier paper 60 at the lower absorbent pad surface 43. Then the device can be easily adhered to the skin at bandaging material 220 skin side adhesive surface 223a and pressure can be put on the flexible release-paper 110 at a pressure point 117 to help to set the adhesive to the skin. This enhancement, which creates an easily to handle bandage, is also applicable to other embodiments. The double-stick adhesive tape 130 and the weakly-adhered backing 410 are functionally quite equivalent as both are meant to hold the device in its folded configuration until deployed and then both are cleanly removed from the non-adhesive back 222 of the bandaging material 220, only in one case, there is an additional weakly-adhered backing 410, possibly polyethylene, between the adhesive tape 130 and the non-adhesive back 222. Here, temporary weakly-adhered backing 410 is affixed to the non-adhesive back 222 of the bandaging material 220 and that weakly-adhered backing 410 is folded, along with the material of the base of the bandaging material 220 to form the deployer hinge 229. The weakly-adhered backing 410 would then be adhesively held in a folded position such that when the device was deployed by pulling the flexible release-paper 110 at the pull-tab 111, the weakly-adhered backing 410 would be peeled off or pulled away from non-adhesive back 222 of the bandaging material 220 so that the deployer hinge 229 would close, that is be deployed and rotate to flat, and that rotation would bring the entire layer of bandage adhesive 223 of the polyurethane, for example, bandaging materials 220 against the skin where the bandaging materials 220 are fully adhered with the absorbent pad 40 covering the procedure site.

Referring now to FIGS. 21a and 21b, embodiments show an alternative folding method, an alternative absorbent pad placement, and an alternative double-sided tape placement. Here, the deployer hinge 229 is offset and the adhesive tape 130 and its 130 adhesive coating bottom 130a are aligned to the backing edge 411 in such a way that when the deployer is deployed, the adhesive tape 130 at the adhesive coating bottom 130a pulls the weakly adhered backing left 410a cleanly away from the bandaging material 220. The adhesive tape 130, or adhesive coating if it has no tape core, is shown as much thicker than it actually would be to emphasize that the adhesive fills the space between the deployer's 400 weakly adhered backing left 410a and weakly adhered backing right 410b and prevents these two backing sections from being separated. Hence, when the pull-tab 111 is pulled, the thin layer, such as polyurethane, of the bandaging material 220 at the skin side adhesive surface 223a stays adhered to the skin and the weakly adhered backing 410 pulls away from the non-adhesive back 222 and stays with the flexible release-paper 110 and the adhesive tape 130. Next, rotation about the deployer hinge 229 occurs to bring the adhesive flap 223b and the absorbent pad 40 to the skin where they remain adhered and the weakly-adhered backing 410 with the flexible release-paper 110 and the adhesive tape 130 are removed as one piece and discarded. An additional feature of this embodiment is visible at the lower edge 224 of the bandaging material 220 which can be seen to be slightly shorter than the weakly-adhered backing 410, the backing end 411 extending beyond the lower edge 224 of the bandaging material 220. In effect, when the device is deployed and the adhesive coating bottom 130a pulls on the weakly-adhered backing 410, the skin side adhesive surface 223a of the bandaging material 220 adheres to the skin and creates a shear that helps to cleanly separate the weakly-adhered backing 410 from the bandaging material 220.

While certain advantageous embodiments have been chosen to illustrate the invention, it will be understood by those skilled in the art that various modifications can be made herein without departing from the scope of the invention. For example, the geometric configurations shown in the illustrative embodiments are generally rectangular and folded once or twice. However, depending upon the type of procedure, the dressing shape, folds, adhesive locations, and materials may be changed in accordance with those needs without diverting from the scope and spirit of the invention. Likewise, dimensions may similarly be varied according to need and still fall well within the scope of the present invention.

I hereby claim:

1. An invasive medical procedure site dressing, comprising:
a. an observation door hingedly attached to a patch base layer;
b. an absorbent layer interposed between a carrier paper and said patch base layer; and
c. a procedure site aperture which passes through co-aligned apertures in said absorbent layer and said patch base layer; wherein
d. said carrier paper is adhesively adhered to said patch base layer and said absorbent layer is smaller than said patch base layer.

2. The invasive medical procedure site dressing of claim 1, wherein said carrier paper contacts a bottom surface of said absorbent layer and a portion of a bottom face of said patch base layer.

3. The invasive medical procedure site dressing of claim 1, wherein said carrier paper is composed of material designed to affix to and release from said adhesive layer.

4. The invasive medical procedure site dressing of claim 1, wherein said patch base layer is composed of a flexible material.

5. The invasive medical procedure site dressing of claim 1, wherein said patch base layer further comprises an adhesive coating on a bottom face of said patch base layer.

6. The invasive medical procedure site dressing of claim 5, wherein said adhesive coating on said patch base layer is of such strength to adhere said site dressing to said patient's skin without requiring an adhesive coating on said absorbent layer.

7. The invasive medical procedure site dressing of claim 1, wherein said absorbent layer is composed of an absorbent material.

8. The invasive medical procedure site dressing of claim 1, wherein said absorbent layer further comprises an adhesive coating on a bottom surface.

9. The invasive medical procedure site dressing of claim 1, wherein said absorbent layer varies in thickness from about 1/16 of an inch to about one (1) inch.

10. The invasive medical procedure site dressing of claim 1, wherein said procedure site aperture through said absorbent layer and said procedure site aperture through said patch base layer are similar in size and shape.

11. The invasive medical procedure site dressing of claim 1, wherein said observation door is composed of a transparent material.

12. The invasive medical procedure site dressing of claim 1, wherein said observation door is convex-shaped.

13. The invasive medical procedure site dressing of claim 1, wherein said observation door is comprised of a flap member and a fixed member joined at a hinge, said fixed member folded under said flap member at said hinge and adhered to said patch base layer with a permanent adhesive.

14. The invasive medical procedure site dressing of claim 13, wherein said observation door is held in an open and undeployed position by a releasable adhesive bead located on said patch base layer such that said flap member of said observation door can be released from said releasable adhesive bead and swiveled on said hinge to a closed and deployed position.

15. The invasive medical procedure site dressing of claim 14, wherein said flap member is further comprised of a positioning tab placed opposite said hinge.

16. The invasive medical procedure site dressing of claim 13, wherein said flap member of said observation door has a transparent window.

17. The invasive medical procedure site dressing of claim 13, wherein said flap member of said observation door is comprised of a gas permeable material with minute pores and a releasable adhesive on said flap member's top surface.

18. The invasive medical procedure site dressing of claim 17, wherein said flap member of said observation door has an absorbent pad mounted to a backing releasably affixed to said flap member with said releasable adhesive.

19. The invasive medical procedure site dressing of claim 18, wherein said absorbent pad is comprised of an absorbent material, such as, but not limited to, cotton or absorbent polyurethane, and wherein said absorbent pad is cut from said absorbent layer such that said absorbent pad is of about a same size and shape as said absorbent layer.

20. The invasive medical procedure site dressing of claim 18, wherein said absorbent pad has a backing with a removal tab.

21. The invasive medical procedure site dressing of claim 20, wherein said removal tab is extended and said absorbent pad is positioned on a lower side of said extended removal tab.

22. The invasive medical procedure site dressing of claim 1, wherein said observation door is covered by an opaque outer door affixed to said observation door, wherein said opaque outer door has a fixed member adhered with a permanent sealant to said patch base layer, said fixed member joining a flap member by a hinge.

23. The invasive medical procedure site dressing of claim 22, wherein said opaque outer door has a releasable adhesive on a surface.

24. The invasive medical procedure site dressing of claim 23, wherein said opaque outer door is comprised of a gas permeable material.

25. The invasive medical procedure site dressing of claim 23, wherein said releasable adhesive is placed on only one edge of said opaque outer door.

26. A preemptive bandage comprising:
a. a carrier paper;
b. a patch base layer;
c. an observation door having a hinge and hingedly affixed to said patch base layer;
d. an absorbent layer positioned between said carrier paper and said patch base layer; and
e. a procedure site aperture formed through said patch base layer and said absorbent layer, said patch base layer and said absorbent layer co-aligned; wherein
f. said carrier paper is adhesively adhered to said absorbent layer and said patch base layer, said patch base layer is positioned on top of said absorbent layer, and said patch base layer extends beyond said absorbent layer.

27. A dressing comprised of a flexible adhesive layer defining an aperture and a domed-shape observation window.

28. The dressing of claim 27, wherein a removable absorbent pad is positioned in said domed-shape observation window.

29. A dressing after-procedure patch comprised of a removable absorbent pad, a flexible adhesive layer, an aperture through said flexible adhesive layer, and a convex-shaped observation window over said aperture.

30. A method of using an invasive medical procedure site dressing, comprising:
a. cleaning a medical procedure site;
b. removing a carrier paper;
c. contacting an absorbent layer directly to skin at said medical procedure site;
d. positioning a procedure site aperture defined by said absorbent layer completely around said medical procedure site such that sufficient open space is available for performance of a medical procedure;
e. performing said medical procedure at said medical procedure site through said procedure site aperture; and
f. placing an observation door in said observation door's closed and deployed position such that said procedure site aperture is covered and protected by a flap member of said observation door.

31. The method of using the invasive medical procedure site dressing of claim 30, further comprising the step of closing said observation door to bring an absorbent pad located on said observation door into contact with said medical procedure site by wedging said absorbent pad into said procedure site aperture, such that said absorbent pad about completely fills said procedure site aperture and no further pressure application or adhesives are required to hold said absorbent pad in place to wick away fluids and tissue exudate and to cover said medical procedure site.

32. The method of using the invasive medical procedure site dressing of claim 31, further comprising opening said observation door, removing said absorbent pad from said observation door by pulling a backing away from said observation door using a removal tab, and closing said observation door.

33. The method of using the invasive medical procedure site dressing of claim 32, further comprising removing said absorbent pad from said observation door by pulling said removal tab left to rotate said observation door over said procedure site aperture with one single movement, whereby said removal tab is extended and said absorbent pad is positioned on a lower side of said removal tab.

34. The method of using the invasive medical procedure site dressing of claim 30, further comprising accessing said medical procedure site by lifting a releasable adhesive on said flap member's top surface and opening said observation door, and closing said observation door after observation by adjusting a positioning tab to facilitate moving said observation door from an open position back to said closed and deployed position

35. The method of using the invasive medical procedure site dressing of claim 30, further comprising opening an opaque outer door positioned on said observation door by lifting said opaque outer door to an open position, viewing said medical procedure site through said observation door when closed, raising said observation door if access to said medical procedure site is necessary, and closing said observation door and said opaque outer door after access and observation.

36. A method of using a preemptive medical bandage, comprising:
a. cleaning a medical procedure site;
b. removing a carrier paper from an absorbent layer of said medical bandage and placing said absorbent layer onto skin such that a procedure site aperture defined by said absorbent layer surrounds said medical procedure site;
c. performing a medical procedure through said procedure site aperture;
d. closing and deploying an observation door such that said procedure site aperture and said medical procedure site are covered and protected by a flap member of said observation door and an absorbent pad of said observation door contacts said medical procedure site and fills said procedure site aperture;
e. opening said observation door by lifting a releasable adhesive on said flap member's top surface for accessing said medical procedure site and removing said absorbent pad using a removal tab;
f. closing and deploying said observation door from an open to a closed position by adjusting a positioning tab;
g. opening an opaque outer door positioned on said observation door for viewing said medical procedure site through said observation door in a deployed position; and
h. closing said opaque outer door after observation of said medical procedure site through said observation door in a deployed position.

37. A preemptive medical procedure site dressing, comprising:
a. a bandage body and a base forming a ring and defining an aperture through said bandage body and said base; and
b. a door with a back surface and a front surface opposing said back surface; wherein
c. a hinge connects said door with said bandage body.

38. The preemptive medical procedure site dressing of claim 37, further comprising a carrier paper coated with a release coating for packaging and shipping said preemptive medical procedure site dressing, wherein said back surface of said door and a bandage body adhesive of said bandage body are removably adhered to said carrier paper.

39. The preemptive medical procedure site dressing of claim 38, wherein said carrier paper is composed of material designed to affix to and release from said back surface and said bandage body adhesive.

40. The preemptive medical procedure site dressing of claim 37, further comprising an absorbent pad adhesively and centrally positioned on said front surface of said door.

41. The preemptive medical procedure site dressing of claim 40, wherein said absorbent pad is of a similar size, shape, and configuration as said aperture, such that when said door is rotated about said hinge toward said bandage body to deploy said preemptive medical procedure site dressing, said absorbent pad may fit into said aperture and slightly protrude through said aperture.

42. The preemptive medical procedure site dressing of claim 40, wherein said absorbent pad is comprised of an absorbent material, such as cotton, natural absorbent fibers, or absorbent polyurethane.

43. The preemptive medical procedure site dressing of claim 40, wherein said absorbent pad varies in thickness from about 1/32 of an inch to about one (1) inch.

44. The preemptive medical procedure site dressing of claim 40, wherein said absorbent pad is positioned on a piece of release-paper backing with a releasable coating, said release-paper backing releasably affixed to said front surface of said door with said releasable coating on a release tab, such that said absorbent pad and said release-paper backing are removable from said preemptive medical procedure site dressing.

45. The preemptive medical procedure site dressing of claim 37, wherein said bandage body and said base are composed of a flexible, non-toxic, and transparent material, such as natural and synthetic polymers, rubber, or polyurethane

46. The preemptive medical procedure site dressing of claim 37, wherein said door is composed of a transparent material

47. The preemptive medical procedure site dressing of claim 37, wherein said door is composed of an opaque material and said door has a transparent window.

48. The preemptive medical procedure site dressing of claim 37, wherein said door is convex-shaped.

49. The preemptive medical procedure site dressing of claim 37, wherein at least a portion of said door is constructed of a gas permeable material, a semi-permeable membrane, or provided with small pores, holes, or vents.

50. The preemptive medical procedure site dressing of claim 37, wherein said front surface of said door further comprises a non-permanent and re-positionable adhesive.

51. The preemptive medical procedure site dressing of claim 37, wherein said aperture is covered by a non-coring, flexible, self-sealing, non-pyrogenic clear or translucent injection material, such as a thermoplastic elastomeric film.

52. The preemptive medical procedure site dressing of claim 51, wherein said injection material is held in place over said aperture by a film adhesive between an underside periphery of said aperture and said base of said bandage body.

53. The preemptive medical procedure site dressing of claim 51, wherein said injection material is held in place by a retaining layer, said retaining layer having an adhesively coated retaining layer bottom attached to a top surface of said injection material at a periphery and to said base of said bandage body to hold said injection material firmly in place on said bandage body.

54. The preemptive medical procedure site dressing of claim 53, wherein said retaining layer has a retaining layer aperture.

55. A preemptive bandage for medical procedures, comprising:
a. a bandage body and a procedure site aperture formed by a 360-degree ring through said bandage body; and
b. a door hingedly attached to said bandage body, such that said door rotates about a hinge and a flexible material to deploy and close over said bandage body.

56. The preemptive bandage for medical procedures of claim 55, further comprising a carrier paper with a release coating upon which a back surface of said door and a bandage body adhesive of said bandage body are removably adhered.

57. The preemptive bandage for medical procedures of claim 55, further comprising an absorbent pad removably and adhesively positioned on a front surface of said door.

58. A preemptive bandage comprising:
a. an absorbent pad;
b. a bandage body and a procedure site aperture formed by a 360-degree ring through said bandage body and a base of said bandage body;
c. a door hingedly connected with said bandage body, such that said door rotates about a hinge to close and cause a front surface of said bandage body to contact said base of said bandage body;
d. a non-coring, flexible, self-sealing, non-pyrogenic clear or translucent injection material held in place over said procedure site aperture by a film adhesive between an underside periphery of said aperture and said base of said bandage body; and
e. a retaining layer positioned over said injection material, said retaining layer having a retaining layer bottom attached to a top surface of said injection material at a periphery and to said base of said bandage body to hold said injection material firmly in place on said bandage body.

59. A method of using a preemptive medical procedure site dressing, comprising:
a. removing a carrier paper from said preemptive medical procedure site dressing;
b. placing said preemptive medical procedure site dressing on a patient's skin so that a procedure site aperture of a bandage body completely surrounds a procedure site with enough open space available for a medical procedure to be performed;
c. adhering a bandage body adhesive of said bandage body to said patient's skin;
d. performing said medical procedure; and
e. closing a door hingedly attached to said bandage body by rotating said door about a hinge to bring an absorbent pad close to or in contact with said procedure site and to bring a front surface of said door into contact with a base of said bandage body to seal said procedure site aperture.

60. The method of using a preemptive medical procedure site dressing of claim 59, further comprising pushing said absorbent pad against a needle barrel and said patient's skin as said needle barrel is being removed from said patient's skin after a needle puncture has been performed.

61. The method of using a preemptive medical procedure site dressing of claim 59, further comprising removing said absorbent pad adhered to a removable release tab by pulling a release-paper backing.

62. The method of using a preemptive medical procedure site dressing of claim 61, further comprising grasping said release tab and rotating said release-paper backing towards said bandage body with said absorbent pad still attached to said paper-release backing.

63. The method of using a preemptive medical procedure site dressing of claim 61, further comprising pulling said release tab away from said door to rotate said door on said hinge to close said door over said procedure site aperture with a single movement and simultaneously removing said release-paper backing and said absorbent pad from said front surface of said door

64. A method of using a preemptive medical bandage, comprising:
a. removing a carrier paper from said preemptive medical bandage and placing said preemptive medical bandage onto a patient's skin such that an aperture defined by a bandage body surrounds a medical procedure site;
b. performing a medical procedure through said aperture;
c. closing and deploying a door hingedly attached to said bandage body, such that said aperture and said medical procedure site are sealed by a front surface of said door contacting a base of said bandage body and an absorbent pad of adhesively attached to said door contacts said medical procedure site and fills said aperture; and
d. removing said absorbent pad adhered to a graspable and removable release tab by pulling a release-paper backing and reclosing said door.

65. A method of using a preemptive bandage with a retaining layer and an injection material, comprising:
a. penetrating with a hypodermic needle on a syringe said retaining layer and said injection material positioned on a bandage body through a procedure site aperture;
b. removing said hypodermic needle on said syringe through said retaining layer and said injection material; and
c. closing a door by rotating said door about a hinge connecting said door with said bandage body.

66. A bandage deployer, comprising:
a. a deployer component; and
b. a bandage component; wherein
c. said deployer component has one or more deployer apertures positioned on a flexible release-paper; and wherein
d. said bandage component has an adhesive flap and a bandage back flap which adhere to each other when said bandage deployer is folded at a hinge to bring said adhesive flap into contact with said bandage back flap through said deployer apertures;
e. a bandaging material on said bandage component; and
f. an absorbent pad adhered to said adhesive flap.

67. The bandage deployer of claim 66, wherein said one or more deployer apertures are positioned near an end of said flexible release-paper.

68. The bandage deployer of claim 66, wherein said flexible release-paper is on a bottom of said deployer component bottom.

69. The bandage deployer of claim 66, wherein said deployer component further has a piece of adhesive tape to hold said bandage deployer in its closed configuration.

70. The bandage deployer of claim 66, wherein said flexible release-paper has an adhesive on a non-coated side of an end of said flexible release-paper.

71. The bandage deployer of claim 66, wherein an end of said flexible release-paper, from a left edge to a right edge, of said deployer component, is coated on at least one side with a re-positionable adhesive.

72. The bandage deployer of claim 66, wherein said flexible release-paper has a plurality of hinge locations on said deployer component.

73. The bandage deployer of claim 66, wherein a bandage adhesive coating of said bandage component adheres said bandage deployer to a carrier paper for packaging and shipment.

74. A method to deploy a bandage deployer, comprising the steps of:
a. bending a flexible release-paper to about 170-degrees in a down direction at a potential left bend to produce a left bend;
b. bending said flexible release paper back about 189-degress at a potential right bend to produce a right bend;
c. positioning an adhesive tape located at an end of said flexible release-paper between a bandage back flap and a bandage front flap on a non-adhesive back of a bandaging material of a bandage component, such that said bandage back flap and said bandage front flap are adhesively apposing and create a deployer hinge;
d. pulling a pull tab at an exposed end of said bandage component by pulling said pull tab toward said left bend; and
e. pulling said pull tab toward said right bend to rotate said bandaging material around said deployer hinge and create shear between an adhesive of said adhesive tape and a release-paper end, thereby deploying said bandage deployer and straightening out a skin side adhesive surface of said bandage component against a patient's skin or bringing an absorbent pad into contact with said patient's skin; wherein said flexible release paper is remaining adhered to a surface of said bandaging material some part of said adhesive tape until said bandaging material is rotated around said release-paper end.

75. A method to deploy a bandage deployer, comprising:
a. positioning a deployer component and a bandage component on a patient's skin with a deployer hinge near an injection site and about half a distance from an absorbent pad left edge and an absorbent pad right edge;
b. inserting a needle;
c. pulling either a pull-tab or a release at a right bend toward said deployer hinge, thereby pulling one side of an adhesive tape bottom off of a bandage back flap of a bandaging material and rotating an absorbent pad around said deployer hinge all the way to needle insertion site and pressing said absorbent pad near said needle insertion site;
d. retracting said needle with said absorbent pad and said bandaging material covering said needle insertion site;
e. pressing an adhesive flap against said needle insertion site;
f. pulling said pull-tab towards an end of said bandaging material;
g. pulling said deployer component away from a non-adhesive back and pulling said adhesive tape off of said non-adhesive back;
h. setting said bandage component by pressing said bandage component against a patient's skin from one end to another.

76. A medical procedure site dressing deployer, comprising:
a. a deployer component having a deployer hinge, a deployer component bottom facing an adhesive flap of a bandaging material, said bandaging material having a non-adhesive back and an adhesive flap, and a flexible release-paper adhesively adhered to said deployer component bottom; and
b. an absorbent pad located around said deployer hinge and bent with said deployer hinge such that said deployer component can be lifted away from a release coating on a carrier paper with a pull-tab without touching a said adhesive flap and an adhesive coating bottom; wherein
c. said absorbent pad traverses said bandaging material and a skin side adhesive surface is adhered to a patient's skin to secure said deployer.

77. A method of deploying a medical procedure site dressing deployer, comprising the steps of:
a. removing a carrier paper from a deployer component;
b. positioning said medical procedure site dressing deployer near a procedure site such that a medical procedure site is about half a distance from an absorbent pad left edge and an absorbent pad right edge;
c. lifting a pull-tab to pull a flexible release-paper coated release-surface of a deployer component bottom away from an adhesive flap of bandaging materials;
d. pulling said flexible release-paper almost completely free of said bandaging materials;
e. rotating said bandaging materials on about a deployer hinge so as to bring a skin-side adhesive flap into contact with a patient's skin and said adhesive flap holding said absorbent pad to cover said procedure site; and
f. pulling said pull-tab such that an adhesive tape is pulled off of the skin thereby freeing said deployer component to be discarded.

78. A deployer for a medical procedure site dressing to preemptively deliver a thin bandaging material, comprising:
a. a deployer component having a deployer hinge, a deployer component bottom adhesively adhered to a carrier paper, and a bandaging material with a weakly-adhered backing bonded to a non-adhesive back;
b. an absorbent pad having a lower absorbent pad surface, whereby said absorbent pad and said deployer hinge are positioned so as to hold said deployer component in its open configuration until deployed; and
c. a flexible release-paper with a pressure point and a double-sided adhesive tape, whereby said adhesive tape attaches said flexible release-paper to said layer of weakly-adhered backing.

79. The deployer for a medical procedure site dressing of claim 78, said bandaging material composed of a clear thin polyurethane bonded with said weakly-adhered backing composed of a polyethylene release material on said non-adhesive back of said bandaging material.

80. The deployer for a medical procedure site dressing of claim 78, wherein said deployer hinge is offset and said adhesive tape and an adhesive coating bottom are aligned to a backing edge, wherein said adhesive tape fills a space between said deployer's weakly adhered backing left and weakly adhered backing right and prevents said weakly adhered backing left and said weakly adhered backing right from being separated

81. The deployer for a medical procedure site dressing of claim 80, wherein a lower edge of said bandaging material is slightly shorter than said weakly-adhered backing, and wherein a backing end extends beyond said lower edge.

82. A method of deploying a deployer for a medical procedure site dressing to preemptively deliver a thin bandaging material, comprising:
a. bending an absorbent pad about 180-degrees in such away that said absorbent pad can be grasped with a first finger and a second finger by folding a weakly-adhered backing and material at a base of said bandaging material to form a deployer hinge;
b. positioning said first finger on a flexible release-paper at a pull tab and positioning said second finger between said absorbent pad and a carrier paper at a lower absorbent pad surface;
c. adhering said deployer to a patient's skin at a skin side adhesive surface of said bandaging material;
d. holding said weakly-adhered backing at an interface in a folded position;
e. pulling said pull-tab to release said flexible release paper;
f. removing said weakly-adhered backing from a non-adhesive back of said bandaging material so that said deployer hinge closes and deploys, thereby rotating to flat and bringing a bandage adhesive of said bandaging materials against said patient's skin where said bandaging materials are fully adhered with said absorbent pad covering said procedure site; and
g. applying pressing on said flexible release-paper at a pressure point to set said skin side adhesive surface to said patient's skin.

83. The method of deploying a deployer for a medical procedure site dressing to preemptively deliver a thin bandaging material of claim 82, further comprising pulling a weakly adhered backing left cleanly away from said bandaging material and stays with said flexible release-paper and said adhesive tape, such that when said pull-tab is pulled, said bandaging material at said skin side adhesive surface stays adhered to said patient's skin, and rotation about said deployer hinge occurs to bring an adhesive flap and said absorbent pad to said patient's skin where said adhesive flap and said absorbent pad will remain adhered and said weakly-adhered backing with said flexible release-paper and said adhesive tape are removed as one piece and discarded.
